(19) United States
(12) Patent Application Publication
(10) Pub. No.: US 2020/0222505 A1
(43) Pub. Date: Jul. 16, 2020

(54) METHODS OF DELAYING AND PREVENTING ACUTE MYELOID LEUKEMIA RELAPSE

(71) Applicant: IMMUNE PHARMACEUTICALS, INC., Englewood Cliffs, NJ (US)

(72) Inventors: Anna Martner, Gothenburg (SE); Fredrik Bergh Thorén, Gothenburg (SE); Johan Aurelius, Gothenburg (SE); Kristoffer Hellstrand, Gothenburg (SE)

(21) Appl. No.: 16/626,844

(22) PCT Filed: Jun. 28, 2018
(86) PCT No.: PCT/US2018/040037

§ 371 Date: Dec. 26, 2019

Related U.S. Application Data
(60) Provisional application No. 62/526,952, filed on Jun. 29, 2017.

(51) Int. Cl. A61K 38/20; A61K 31/417; C12Q 1/6886

(52) U.S. Cl. CPC A61K 38/2013; A61K 45/06; C12Q 1/6886; A61K 31/417

(57) ABSTRACT

Disclosed herein include methods of delaying and preventing acute myeloid leukemia (AML) relapse in patients with mutant nucleophosmin 1 (NPM1) by co-administration of histamine, or derivatives thereof, and interleukin-2 (IL-2). In some embodiments, an unexpected delay in and/or prevention of relapse results in a surprising increase in leukemia- free survival rate and overall survival rate.

### CROSS REFERENCE TO RELATED APPLICATIONS

The present application claims priority under 35 U.S.C. § 119(e) to U.S. Provisional Application No. 62/526,952, filed on Jun. 29, 2017. The content of this related application is herein expressly incorporated by reference in its entirety.

### BACKGROUND

### Field

The present application relates to the fields of pharmaceutical chemistry, biochemistry and medicine. One aspect relates to the prevention and/or delay of the onset of relapse to acute myeloid leukemia (AML) in AML patients, for example patients in complete remission (CR) from AML, by administration of histamine or derivatives thereof, and IL-2.

### Description of Related Art

AML is a genetically and morphologically heterogeneous cancer characterized by a clonal expansion of immature myeloid cells in bone marrow and other organs. Most patients with AML achieve microscopic disappearance of leukemic cells (e.g. complete remission, CR) after initial rounds of chemotherapy (induction), which are typically given immediately after diagnosis. The standard treatment in AML comprises additional rounds of chemotherapy (consolidation) aiming at eliminating residual leukemic cells. Despite this intensive treatment, as many as >60% of adult patients will experience relapse of leukemia within 2-3 years with poor prognosis for survival. Relapse is a significant reason why the 5-year survival rate in adult AML remains in the range of 25-30% (Burnett et al., J Clin Oncol. 2011 Feb. 10;29(5):487-94).

Nucleophosmin (NPM, aka B23 or numatrin) is a 35-50 kD phosphoprotein found at high levels in the granular regions of nucleoli. There are at least two isoforms of NPM, NPM1 and NPM1.2. While the precise cellular functions of NPM remain to be determined, nuclear NPM is thought to play a significant role in the formation of ribosomes. NPM is also found to be shuttled between the nucleus and the cytoplasm, presumably to assist in transport of proteins to the nucleus along with preventing protein aggregation and degradation (Falini et al., Blood. 2007 Feb. 1;109(3):874-85).

The gene encoding NPM1, located on chromosome 5, is mutated in leukemic cells in approximately 30% of patients with AML. The functional consequence of the most prevalent NPM1 mutations is that NPM1 is retained in the cytoplasm, which results in aberrant cytoplasmic accumulation of mutated NPM1 (NPMc). Mutated NPM1 with ensuing accumulation of NPMc is more commonly observed in myelomonocytic (FAB class M4) and monocytic (FAB class M5) forms of AML and in patients with a normal karyotype (AMLNK) in leukemic cells. The incidence of NPM1 mutations has been observed to increase with age, and as many as 50-60% of adult patients with AML-NK harbor leukemic cells with mutated NPM1 (Falini et al., Blood. 2007 Feb. 1;109(3):874-85).

AML with mutated NPM1 is typically associated with a more favorable prognosis compared with other forms of AML, in particular when leukemic cells harbor mutated NPM1 in the absence of other genetic aberrations, including mutated FLT3. However, patients harboring NPM1-mutated transcripts in blood after the completion of chemotherapy (induction and consolidation, cf. above) show distinctly higher risk of relapse and death. Ivey et al. thus reported that AML patients with presence of NPM1-mutated transcripts in blood after chemotherapy showed high relapse risk of and poor overall survival compared with patients in whom such transcripts were not detected (Ivey et al., N Engl J Med. 2016 Feb. 4;374(5):422-33).

Histamine dihydrochloride is derived from the biogenic amine histamine. It suppresses the production of reactive oxygen species that inhibits the functions of T cells and natural killer (NK) cells, including their responsiveness to immune activating cytokines. Co-administration of the cytokine interleukin (IL)-2 and histamine dihydrochloride assists the activation of T cells and NK cells by IL-2, leading to the destruction of cancer cells, including those of acute myeloid leukemia (AML) Immunotherapy with histamine dihydrochloride (HDC) in conjunction with the T- and NK-cell activating cytokine interleukin-2 (HDC/IL-2) gained approval for relapse prevention in AML throughout the EU in 2008.

The prospect of long-term survival after relapse of AML is poor, and NPM1-mutated AML is a distinct leukemia entity that accounts for one third of cases of AML in adults. There is a long-felt yet unmet need for effective treatments for delaying and preventing AML relapse, including relapse of NPM1-mutated AML.

### SUMMARY

Disclosed herein include methods and compositions for improving a survival rate of patients having acute myeloid leukemia (AML). In some embodiments, the method comprises (a) identifying the presence of mutant nucleophosmin 1 (NPM1) in a patient having AML; and (b) administering to a patient identified as having a mutant NPM1 in step (a) a therapeutically effective amount of IL-2 and a therapeutically effective amount of an agent selected from the group consisting of histamine, a histamine structural analog having H₂-receptor activities, an endogenous histamine releasing preparation, a non-histamine derivative H₂-receptor agonist, and a combination thereof. In some embodiments, the method comprises (a) acquiring knowledge of the presence of one or more molecular alterations in a biological sample from an AML patient, wherein said one or more molecular alterations comprises the presence of mutant NPM1; and (b) for a patient known to have a mutant NPM1 in step (a), administering to the patient a therapeutically effective amount of IL2 and a therapeutically effective amount of an agent selected from the group consisting of histamine, a histamine structural analog having H₂-receptor activities, an endogenous histamine releasing preparation, a non-histamine derivative H₂-receptor agonist, and a combination thereof. In some embodiments, administration of IL-2 and the agent results in an increase in a survival rate of the treated patients compared to the untreated patients. In some embodiments, the survival rate is leukemia-free survival rate. In some embodiments, the survival rate is overall survival rate. In some embodiments, the administration of IL-2 and the agent results in an increase of at least 30% in a survival rate of treated patients compared to the untreated patients. In some embodiments, the administration of IL-2 and the agent results in an increase of at least 30% in a survival rate of treated patients compared to the untreated patients. In some embodiments, the administration of IL-2 and the agent results in an increase of at least 50% in a survival rate of treated patients compared to the untreated patients. In some embodiments, the administration of IL-2 and the agent results in an increase of the patient's LFS and/or OS time by at least 1.1 fold (e.g, 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, or, 5 fold, and overlapping ranges) or more relative to the duration of LFS and/or OS of the untreated patients.

Further disclosed herein include methods and compositions for preventing and/or delaying the onset of relapse to AML in a patient in complete remission (CR) from AML. In some embodiments, the method comprises the steps of: (a) identifying the presence of mutant NPM1 in a patient in CR from AML; and (b) administering to a patient identified as having a mutant NPM1 in step (a) a therapeutically effective amount of IL2 and a therapeutically effective amount of an agent selected from the group consisting of histamine, a histamine structural analog having H₂-receptor activities, an endogenous histamine releasing preparation, a non-histamine derivative H₂-receptor agonist, and a combination thereof. In some embodiments, the method comprises (a) acquiring knowledge of the presence of one or more molecular alterations in a biological sample from an AML patient, wherein said one or more molecular alterations comprises the presence of mutant NPM1; and (b) for a patient known to have a mutant NPM1 in step (a), administering to the patient a therapeutically effective amount of IL2 and a therapeutically effective amount of an agent selected from the group consisting of histamine, a histamine structural analog having H₂-receptor activities, an endogenous histamine releasing preparation, a non-histamine derivative H₂-receptor agonist, and a combination thereof. In some embodiments, the administration of IL-2 and the agent prevents and/or delays the onset of relapse to AML in said patient. In some embodiments, relapse comprises at least 5% blast cells in the bone marrow. In some embodiments, relapse comprises extramedullary leukemia. In some embodiments, the administration of IL-2 and the agent delays relapse of AML of treated patients by at least 1 week (e.g., 7 days, 10 days, 30 days, 2 months, 3 months, 4 months, 5 months, 6 months, 7 months, 8 months, 9 months, 10 months, 11 months, 1 year, 18 months, 2 years, 30 months, 3 years, 40 months, 4 years, 5 years, 6 years, 7 years, 8 years, 9 years, 10 years, 15 years, 20 years, 25 years, 30 years, 35 years, 40 years, 50 years, 55 years, 60 years, 65 years, 70 years, 75 years, and overlapping ranges) compared to the untreated patients. In some embodiments, the administration of IL-2 and the agent delays relapse of AML of treated patients by at least 3 months compared to the untreated patients. In some embodiments, the administration of IL-2 and the agent delays relapse of AML of treated patients by at least 6 months compared to the untreated patients. In some embodiments, the administration of IL-2 and the agent delays relapse of AML of treated patients by at least 12 months compared to the untreated patients.

Further disclosed herein include methods of prolonging remission from AML, comprising the steps of: (a) identifying the presence of mutant NPM1 in a patient in remission from AML; and (b) administering to a patient identified as having a mutant NPM1 in step (a) a therapeutically effective amount of IL2 and a therapeutically effective amount of an agent selected from the group consisting of histamine, a histamine structural analog having H₂-receptor activities, an endogenous histamine releasing preparation, a non-histamine derivative H₂-receptor agonist, and a combination thereof. In some embodiments, the method comprises (a) acquiring knowledge of the presence of one or more molecular alterations in a biological sample from an AML patient, wherein said one or more molecular alterations comprises the presence of mutant NPM1; and (b) for a patient known to have a mutant NPM1 in step (a), administering to the patient a therapeutically effective amount of IL2 and a therapeutically effective amount of an agent selected from the group consisting of histamine, a histamine structural analog having H₂-receptor activities, an endogenous histamine releasing preparation, a non-histamine derivative H₂-receptor agonist, and a combination thereof. In some embodiments, the administration of IL-2 and the agent prolongs remission from AML in said patient. In some embodiments, the administration of IL-2 and the agent prolongs remission from AML of the treated patients by at least 1 week (e.g., 7 days, 10 days, 30 days, 2 months, 3 months, 4 months, 5 months, 6 months, 7 months, 8 months, 9 months, 10 months, 11 months, 1 year, 18 months, 2 years, 30 months, 3 years, 40 months, 4 years, 5 years, 6 years, 7 years, 8 years, 9 years, 10 years, 15 years, 20 years, 25 years, 30 years, 35 years, 40 years, 50 years, 55 years, 60 years, 65 years, 70 years, 75 years, and overlapping ranges) compared to the untreated patients. In some embodiments, the administration of IL-2 and the agent prolongs remission from AML of the treated patients by at least 3 months compared to the untreated patients. In some embodiments, the administration of IL-2 and the agent prolongs remission from AML of treated patients by at least 6 months compared to the untreated patients. In some embodiments, administration of IL-2 and the agent prolongs remission from AML of treated patients by at least 12 months compared to the untreated patients.

In some embodiments, the method comprises administering the agent twice a day. In some embodiments, the agent is administered in an amount of about 0.1 mg/day to about 10 mg/day (e.g., 0.1 mg/day, 0.2 mg/day, 0.4 mg/day, 0.6 mg/day, 0.8 mg/day, 1.0 mg/day, 1.5 mg/day, 2.0 mg/day, 2.5 mg/day, 3.0 mg/day, 3.5 mg/day, 4.0 mg/day, 4.5 mg/day, 5.0 mg/day, 5.5 mg/day, 6.0 mg/day, 6.5 mg/day, 7.0 mg/day, 7.5 mg/day, 8.0 mg/day, 8.5 mg/day, 9.0 mg/day, 9.5 mg/day, 10.0 mg/day, and overlapping ranges). In some embodiments, the agent is histamine In some embodiments, the agent is histamine dihydrochloride. In some embodiments, the agent is histamine diphosphate. In some embodiments, the agent is the N-methyl-histamine. In some embodiments, the N-methyl-histamine comprises Na-methyl-histamine dihydrochloride (NMH). In some embodiments, the histamine is administered at 0.5 mg twice a day. In some embodiments, the method comprises administering IL-2 twice a day. In some embodiments, the IL-2 can be administered in an amount of about 5,000 U/kg/day to about 300,000 U/kg/day (e.g, 5,000 U/kg/day, 6,000 U/kg/day, 8,000 U/kg/day, 10,000 U/kg/day, 15,000 U/kg/day, 25,000 U/kg/day, 50,000 U/kg/day, 100,000 U/kg/day, 200,000 U/kg/day, 300,000 U/kg/day, and overlapping ranges). In some embodiments, IL-2 is administered at a dosage of 16,400 U/kg twice a day. In some embodiments, the agent and IL-2 are administered on the same days. In some embodiments, the agent and IL-2 are administered together. In some embodiments, the administration of the agent and said IL-2 is performed simultaneously. In some embodiments, the agent and IL-2 are administered separately. In some embodiments, the administration of the agent and the administration of IL-2 are performed within 24 hours.

In some embodiments, the administration of the agent and/or IL-2 is accomplished by one or more of intramuscular injection, subcutaneous injection, intradermal injection, intravenous injection, implantation infusion device, inhalation, and transdermal diffusion. In some embodiments, the administration of the agent and/or IL-2 is accomplished by subcutaneous injection.

In some embodiments, the method comprises administrating the agent and IL-2 are once per day. In some embodiments, the agent and IL-2 are administered for at least one cycle. In some embodiments, the agent and IL-2 are administered for at least two cycles. In some embodiments the agent and IL-2 are administered for at least six cycles. In some embodiments, one cycle comprises at least 2 (for example, 2, 3, 4, 5, 6, 7, 8, 10, 12, 14, 16, 18, 20, 22, 24, 26, 28, 30, 35, 40, and ranges in-between) consecutive days of treatment. In some embodiments, one cycle comprises 21 consecutive days of treatment. In some embodiments, an interval between two treatment cycles is at least two (for example, 2, 3, 4, 5, 6, 7, 8, 10, 12, 14, 16, 18, 20, 22, 24, 26, 28, 30, 35, 40, and ranges in between) days. In some embodiments, an interval between two treatment cycles is at least two weeks. In some embodiments, an interval between two treatment cycles is at least three weeks. In some embodiments, an interval between two treatment cycles is at least six weeks.

In some embodiments, the patient is in complete remission (CR) from AML. In some embodiments, complete remission comprises less than 5% blast cells in normocellular bone marrow and/or an absence of extramedullary leukemia. In some embodiments, the patient has a de novo AML. In some embodiments, the patient has a secondary AML. In some embodiments, the patient has recurrent, relapsing or refractory AML. In some embodiments, the recurrent or relapsing AML is caused by minimal residual disease (MRD) and/or leukemic stem cells. In some embodiments, the patient's leukemic cells have a normal karyotype. In some embodiments, the patient has already undergone 2 or more rounds of chemotherapy. In some embodiments, the patient has already undergone 4 or more rounds of chemotherapy. In some embodiments, the patient is undergoing immunotherapy for relapse prevention. In some embodiments, the patient has experienced a partial response or complete response, is in remission, is asymptomatic, has a low number of abnormal cells and/or has a non-detectable disease based on one or more of the following: (i) a total body leukemia burden below approximately 10⁹ cells and/or less than 5% blasts in the marrow and/or no signs or symptoms of leukemia; (ii) a greater than 25% reduction in the serum protein M level; (iii) a greater than 50% reduction in the serum protein M level; (iv) 10% or more plasma cells in the bone marrow, but does not meet the criteria for multiple myeloma (MM); (v) serum M proteins levels greater than or equal to 3 g/dL; (vi) 10% or more plasma cells in the bone marrow with no evidence of end-organ damage; (vii) serum M protein levels greater than or equal to 3 g/dL and has 10% or more plasma cells in the bone marrow; (viii) serum M protein levels greater than or equal to 3 g/dL and has 10% or more plasma cells in the bone marrow and no evidence of end-organ damage; and (ix) less than 10% plasma cells in the bone marrow.

In some embodiments, the patient has completed induction chemotherapy. In some embodiments, the patient is a patient who relapses from complete remission of AML after induction chemotherapy. In some embodiments, the patient has completed induction and consolidation chemotherapy. In some embodiments, administration of IL-2 and the agent begins the same day after consolidation chemotherapy is completed. In some embodiments, administration of IL-2 and the agent begins the same day after consolidation chemotherapy is completed. In some embodiments, administration of IL-2 and the agent begins between about 1 day (e.g., 24 hours, 36 hours, 48 hours, 3 days, 4 days, 5 days, 6 days, one week, 10 days, 12 days, two weeks, three weeks, one month, 6 weeks, 2 months, 4 months, 6 months, 8 months, 10 months, 12 months, 14 months, and ranges in between) after consolidation chemotherapy is completed. In some embodiments, administration of IL-2 and the agent begins between about 1 day and about 300 days after consolidation chemotherapy is completed. In some embodiments, administration of IL-2 and the agent begins about 200 days after consolidation chemotherapy is completed. In some embodiments, administration of IL-2 and the agent begins about 100 days after consolidation chemotherapy is completed. In some embodiments, administration of IL-2 and the agent begins about 50 days after consolidation chemotherapy is completed.

In some embodiments disclosed herein, the presence of the mutant NPM1 is determined by identifying a patient nucleic acid encoding the mutant NPM1. In some embodiments the patient nucleic acid is genomic DNA and/or mRNA. In some embodiments, the patient nucleic acid is obtained from an acellular body fluid (e.g., serum and/or plasma) of said patient. In some embodiments, identifying a patient nucleic acid encoding the mutant NPM1 comprises amplification of at least a portion of exon 12 of NPM1. In some embodiments, the amplification comprises polymerase chain reaction (PCR), such as, for example, real-time PCR (RT-PCR). In some embodiments, identifying a patient nucleic acid encoding the mutant NPM1 comprises using an oligonucleotide probe complimentary to a portion of exon 12 of NPM1. In some such embodiments, the oligonucleotide probe comprises a label (e.g., a fluorescent label).

In some embodiments, the mutant NPM1 comprises one or more mutations in exon 12 NPM1 that cause cytoplasmic location of NPM1 protein. For example, in some embodiments, the mutant NPM1 comprises one or more of the NPM1 mutations selected from the group consisting of: Mutation A, Mutation B, Mutation C, Mutation D, Mutation E, Mutation F, Mutation E*, Mutation G*, Mutation H*, Mutation J, Mutation L, Mutation K, Mutation M, Mutation N, Mutation O, Mutation P, Mutation Q, Mutation Gm, Mutation Km, Mutation Lm, Mutation Nm, Mutation Om, Mutation Qm, Mutation 1, Mutation 3, Mutation 4, Mutation 6, Mutation 7, Mutation 12, Mutation 13, Mutation 10, Mutation 14, Mutation G+, Mutation H+, Mutation I+, Mutation J+, Mutation I, and a combination thereof. In some embodiments, the mutant NPM1 comprises one or more of the following NPM1 mutations: Mutation A, Mutation B, Mutation C, Mutation D, Mutation E or Mutation F. In some embodiments, the mutant NPM1 comprises a signal motif of nuclear export (NES) in exon 12 of NPM1. In some such embodiments, the NES comprises the amino acid sequence YxxxYxxYxY, wherein Y is a hydrophobic amino acid selected from the group consisting of leucine, isoleucine, methionine, valine, phenylalanine, and wherein x can be any amino acid.

Also disclosed herein include methods of acquiring knowledge of the presence of one or more molecular alterations in a biological sample from an AML patient. In some embodiments, said one or more molecular alterations comprises the presence of mutant NPM1. In some embodiments, knowledge of the presence of mutant NPM1 is acquired from an analytical assay, such as, for example, nucleic acid sequencing, polypeptide sequencing, restriction digestion, capillary electrophoresis, nucleic acid amplification-based assays, nucleic acid hybridization assay, comparative genomic hybridization, real-time PCR, quantitative reverse transcription PCR (qRT-PCR), PCR-RFLP assay, HPLC, mass-spectrometric genotyping, fluorescent in-situ hybridization (FISH), next generation sequencing (NGS), a kinase activity assay, and any combination thereof. In some embodiments, knowledge of the presence of mutant NPM1 is acquired from an antibody-based assay, such as, for example, ELISA, immunohistochemistry, western blotting, mass spectrometry, flow cytometry, protein-microarray, immunofluorescence, a multiplex detection assay, or any combination thereof. In some embodiments, knowledge of the presence of mutant NPM1 is acquired from immunohistochemistry.

In some embodiments, the presence of the mutant NPM1 is determined by identifying mutant NPM1 protein in patient cells. For example, the mutant NPM1 protein can be identified in the cells by identifying NPM1 protein in cytoplasm of the cells. In some such embodiments, the mutant NPM1 protein is identified in cytoplasm of the cells immunohistochemically. In some embodiments, the mutant NPM1 protein is identified in the cells with an antibody that selectively binds to the mutant NPM1 protein but not a wild-type NPM1 protein.

### BRIEF DESCRIPTION OF THE FIGURES

FIGS. 1A-1B depict data related to the outcome of 22 Phase IV patients diagnosed with NPM1⁺ AML with a NPM1-mutation classified as MRD positive or negative before the onset of treatment with HDC/IL-2 in terms of leukemia-free survival (LFS) and overall survival (OS), respectively.

FIGS. 2A-2B depict data related to the outcome of all patients diagnosed with NPM1⁺ AML (with no other genetic aberrations) in terms of leukemia-free survival (LFS) and overall survival (OS), respectively.

FIGS. 3A-3F depict Kaplan-Meier curves showing days to MRD switch. FIGS. 3A-3C depict Kaplan-Meier curves showing days to MRD switch from negative to positive for patients diagnosed with NPM1⁺ AML without landmark analysis (FIG. 3A), in the 6-months landmark analysis (FIG. 3B), and the 12-months landmark analysis (FIG. 3C), respectively. FIGS. 3D-3F depict Kaplan-Meier curves showing days to MRD switch from negative to positive for patients diagnosed with NPM1⁺ AML that did not receive low dose chemotherapy as maintenance without landmark analysis (FIG. 3D), in the 6-months landmark analysis (FIG. 3E), and the 12-months landmark analysis (FIG. 3F), respectively.

FIGS. 4A-4F depict Kaplan-Meier curves showing days to MRD switch. FIGS. 4A-4C depict Kaplan-Meier curves showing days to MRD switch from negative to positive for patients below 60 years of age diagnosed with NPM1⁺ AML without landmark analysis (FIG. 4A), in the 6-months landmark analysis (FIG. 4B), and the 12-months landmark analysis (FIG. 4C), respectively. FIGS. 4D-F show results corresponding to FIGS. 4A-C in patients diagnosed with NPM1⁺ AML below 60 years of age that did not receive low-dose chemotherapy.

### DETAILED DESCRIPTION

In the following detailed description, reference is made to the accompanying drawings, which form a part hereof. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, can be arranged, substituted, combined, and designed in a wide variety of different configurations, all of which are explicitly contemplated and make part of this disclosure.

AML, also known as acute myeloid leukemia or acute myelogenous leukemia, is a common acute leukemia in adults. In general, the treatment of AML in adults begins with induction therapy using combinations of cytostatic drugs, such as anthracyclines and cytarabine (also known as arabinofuranosyl cytidine or Ara-C), which results in complete remission (CR) in most patients. The induction phase of treatment is followed by intensive consolidation chemotherapy, usually in the form of high-dose cytarabine. Despite the induction and consolidation therapies, less than one third of adult patients with AML who have achieved CR are permanently cured, and an overriding clinical problem is the high rate of leukemic relapse.

Heterogeneity is a characteristic trait of cancer. As a result, the effectiveness of cancer therapy varies significantly among patients. Some cancer therapies may only work specifically on certain patient population. Often times, for a particular cancer treatment, some patients may benefit, some may show little response, and certain population of patients may suffer severe side effects without receiving much real benefits. Therefore, it is important to understand different stages and different sub-types of a cancer disease, such as AML, for developing more effective and individualized treatment for cancer.

In view of the high incidence of leukemic relapse along with the limited prospects of long-term survival after a relapse, there is a need for novel therapeutic strategies to prolong or maintain CR in patients. Moreover, the heterogeneity in cancers calls for better understanding of diverse responses to cancer therapies in patients. NPM1-mutated acute myeloid leukemia (AML) is a distinct leukemia entity that accounts for one third of cases of AML in adults. The present application arose from the unexpected findings that a combination treatment using interleukin-2 (IL-2) along with an agent such as histamine dihydrochloride is surprisingly highly efficacious in treating patients with particular types of AML, such as NPM1-mutated AML. Several embodiments of the present invention relate to unique methods of delaying or preventing AML relapse in a subset of AML patients by the combination treatment of IL-2 and the agent disclosed herein. In several embodiments, the methods described herein provides one or more of the following advantages: (i) increased leukemia-free survival; (ii) increased overall survival; (iii) delay in switch from MRD negative to MRD positive; (iv) delay in reappearance of leukemic cells in blood or bone marrow; and (v) prolonged remission from AML.

### Definitions

As used herein, the term “subject” shall be given its ordinary meaning and shall also refer to all members of the animal kingdom including mammals, and suitably refers to humans. Optionally, the term “subject” includes mammals that have been diagnosed with cancer or are in remission. In some embodiments, the term “subject” refers to a human having, or suspecting of having, a hematological cancer. In some embodiments, the term “subject” refer to a human having AML or suspected of having AML, optionally recurrent or relapsing AML. The terms, “patient” and “subject” are used interchangeably herein.

The term “leukemia” shall be given its ordinary meaning and shall also refer to any disease involving the progressive proliferation of abnormal leukocytes found in hematopoietic tissues, other organs and usually in the blood in increased numbers. “Leukemic cells” refers to leukocytes characterized by an increased abnormal proliferation of such cells.

As used herein, “acute myeloid leukemia” encompasses all forms of acute myeloid leukemia and related neoplasms according to the World Health Organization (WHO) classification of myeloid neoplasms and acute leukemia, including all of the following subgroups in their relapsed or refractory state: Acute myeloid leukemia with recurrent genetic abnormalities, such as AML with t(8;21)(q22;q22); RUNX 1-RUNX m, AML with inv(16)(p 1 3.1 q22) or t(16; 16)(p13.1;q22); CBFB- MYH 1 1, AML with t(9; 1 I)(p22;q23); MLLT3-MLL, AML with t(6;9)(p23;q34); DEK-NUP214, AML with inv(3)(q21 q26.2) or t(3;3)(q21;q26.2); RPN I -EVI 1, AML (megakaryoblastic) with t(1; 22)(p13;q13); RBM15-MKL 1, AML with mutated NPM1 , AML with mutated CEBPA; AML with myelodysplasia-related changes; therapy-related myeloid neoplasms; AML, not otherwise specified, such as AML with minimal differentiation, AML without maturation, AML with maturation, acute myelomonocytic leukemia, acute monoblastic/monocytic leukemia, acute erythroid leukemia (e.g., pure erythroid leukemia, erythroleukemia, erythroid/myeloid), acute megakaryoblastic leukemia, acute basophilic leukemia, acute panmyelosis with myelofibrosis; myeloid sarcoma; myeloid proliferations related to Down syndrome, such as transient abnormal myelopoiesis or myeloid leukemia associated with Down syndrome; and blastic plasmacytoid dendritic cell neoplasm.

As used herein, “chronic myeloid leukemia” (“CML”) refers to a cancer characterized by the increased and unregulated growth of predominantly myeloid cells in the bone marrow and the accumulation of these cells in the blood.

In some embodiments, the methods described herein provide for the treatment of cancer. The terms “treating” or “treatment” shall be given its ordinary meaning and shall also refer to an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease (e.g. maintaining a patient in remission), preventing disease or preventing spread of disease, delay or slowing of disease progression, amelioration or palliation of the disease state, diminishment of the reoccurrence of disease, and remission (whether partial or total), whether detectable or undetectable. “Treating” and “Treatment” can also mean, in some embodiments provided herein, prolonging survival as compared to expected survival if not receiving treatment. “Treating” and “treatment” as used herein also include, in some embodiments, prophylactic treatment. In some embodiments, treatment methods comprise administering to a patient a therapeutically effective amount of IL-2 and an agent as described herein and optionally consists of a single administration, or alternatively comprises a series of administrations.

As used herein, the terms “prevent,” “preventing” and “prevention” and the like, shall be given their ordinary meaning and shall also contemplate an action that occurs before a patient begins to suffer from the regrowth of the cancer and/or which inhibits or reduces the severity of the cancer.

### Patient Populations

In some embodiments, a patient treated by the methods disclosed herein has or is suffering from AML. In some embodiments, a patient treated by the methods disclosed herein is in remission from AML. In some embodiments, the patient has a de novo AML. In some embodiments, the patient has a secondary AML. In some embodiments, a patient treated by the methods disclosed herein is in complete remission (CR) of AML. In some embodiments, complete remission is defined by one or more of the following criteria: (i) normal values for absolute neutrophil count and platelet count, and independence from red cell transfusion; (ii) a bone marrow biopsy that reveals no clusters or collections of blast cells and extramedullary leukemia is absent; (iii) a bone marrow aspiration reveals normal maturation of all cellular components (i.e., erythrocytic, granulocytic, and megakaryocytic); (iv) less than 5% blast cells are present in the bone marrow, and none have a leukemic phenotype; (v) absence of previously detected clonal cytogenetic abnormality confirms the morphologic diagnosis of complete remission. In some embodiments, complete remission (CR) is defined as less than 5% blast cells in normocellular bone marrow, without evidence of extramedullary leukemia. In some embodiments, the patient is one that has complete remission with insufficient hematological recovery. In some embodiments, IL-2 and an agent disclosed herein are administered to a patient in complete remission as defined by one or more of the criteria above and repeated periodically as needed to prevent relapse disease.

In some embodiments, a patient treated by the methods disclosed herein has a measurable amount of minimal residual disease (MRD). As used herein, the term “minimal residual disease” (MRD) shall be given their ordinary meaning and shall also refer to small numbers of cancer cells (such as leukemic cells) that remain in the patient during treatment, or after treatment when the patient is in remission (no symptoms or signs of disease). In some embodiments, MRD is undetectable using conventional diagnostic techniques such as X ray, CT scan, or MRI, or techniques that detect tumors detectable by X ray, CT scan or MRI. MRD can be detected using cell-based detection techniques (such as, for example, immunofluorescence, FACS analysis, or in situ hybridization) or biochemical/molecular biological techniques (such as RT-PCR). In some embodiments, IL-2 and the agent disclosed herein are administered prior to or at the very earliest detection of MRD and repeated periodically as needed to prevent and/or delay relapse to AML. In some embodiments, a patient is considered to suffer from leukemic relapse when there are at least 20% blast cells in the patient's bone marrow or if the patient has extramedullary leukemia.

In some embodiments, a patient treated by the methods disclosed herein is suffering from refractory or relapsed acute myeloid leukemia. As used herein, “relapsed acute myeloid leukemia” is defined as reappearance of leukemic blasts in the blood or greater than 5% blasts in the bone marrow after complete remission not attributable to any other cause. For patients presenting with relapsed AML, more than 5% blasts on baseline bone marrow assessment is required in some embodiments. As used herein, “refractory acute myeloid leukemia” is defined as a failure to achieve a complete remission or complete remission with incomplete blood recovery after previous therapy. Any number of prior anti-leukemia schedules is allowed. In some embodiments, “complete remission” is defined as morphologically leukemia free state (i.e. bone marrow with less than 5% blasts by morphologic criteria and no Auer rods, no evidence of extramedullary leukemia) and absolute neutrophil count greater than or equal to 1,000/μl, and platelets greater than 100,000/μl. As used herein, “complete remission with incomplete blood recovery” is defined as morphologically leukemia free state (i.e., bone marrow with less than 5% blasts by morphologic criteria and no Auer rods, no evidence of extramedullary leukaemia) and neutrophil count less than 1,000/μl, or platelets less than 100,000/μl in the blood.

In some embodiments, the combination treatment of IL-2 and the agent disclosed herein can be performed on a patient with normal karyotype. In other embodiments, the combination treatment of IL-2 and the agent disclosed herein can be performed on a patient with abnormal karyotype. In some embodiments, the combination treatment of IL-2 and the agent disclosed herein can be performed on a patient predicted with good-prognosis, e.g., a patient after the treatment of high-dose AraC. In other embodiments, the combination treatment of IL-2 and the agent disclosed herein can be performed on a patient predicted with poor-prognosis.

In some embodiments, a patient treated by the methods disclosed herein has been treated with surgery, chemotherapy, radiation therapy, a targeted therapy, including therapies that are intended to boost immune system responses against cancer, or a combination thereof. In some embodiments, the AML is resistant to treatment with chemotherapy. For example, in some embodiments the cancer is chemotherapy-resistant AML. In some embodiments, the methods described herein are for the treatment of a patient with recurring or relapsing AML. In some embodiments, relapsing AML caused by minimal residual disease (MRD) and/or leukemic stem cells. In some embodiments, the patient is not in complete remission. For example, in some embodiments the patient has one or more detectable leukemic cells. In some embodiments, the patient has previously undergone chemotherapeutic treatment for cancer but the cancer cells do not respond to the chemotherapy treatment (i.e. refractory cancer). In some embodiments, the patient has previously underdone chemotherapeutic treatment for cancer and has one or more detectable cancer cells. In some embodiments, the patient has not previously undergone chemotherapeutic treatment for cancer.

In some embodiments, IL-2 and an agent disclosed herein are administered to a patient after cessation of another cancer therapy (e.g., a primary cancer therapy), such as chemotherapy, radiation therapy and/or surgery. In some embodiments, the patient has minimal residual disease after the primary cancer therapy (e.g., chemotherapy, radiation therapy and/or surgery).

In some embodiments, a patient treated by the methods disclosed herein has failed a prior therapy for the treatment of AML such as chemotherapy or radiation and is now in remission. In some embodiments, a patient treated by the methods disclosed herein is in first complete remission (CR1). In some embodiments, a patient treated by the methods disclosed herein is in second complete remission (CR2). In some embodiments, a patient treated by the methods disclosed herein is in third complete remission (CR3). In some embodiments, a patient treated by the methods disclosed herein is in fourth complete remission (CR4).

In some embodiments, a patient treated by the methods disclosed herein has undergone induction therapy. In some embodiments, induction chemotherapy comprises cytarabine and/or daunorubicin. In some embodiments, a patient treated by the methods disclosed herein has relapsed from complete remission of AML after receiving an induction chemotherapy treatment regimen.

In some embodiments, a patient treated by the methods disclosed herein has undergone a conditioning regimen. In some embodiments, conditioning regimen is myeloablative. Myeloablative conditioning regimen ablates the cells in the bone marrow, including the AML cells and is usually carried out by total body irradiation (TBI), administration of a cyclophosphamide, administration of busulfan, or combinations thereof. Exemplary cyclophosphamides include endoxan, Cytoxan, neosar, procytox, revimmune, and cycloblastin. In some embodiments, the conditioning regimen is non-myeloablative, i.e., reduced intensity conditioning (RIC). RIC regimen includes doses of chemotherapies and/or radiation lower than myeloablative therapy. Thus, an RIC regimen is considered a gentler regimen that does not eradicate all bone marrow cells and can be used in patients such as the elderly that cannot undergo a myeloablative conditioning regimen.

In some embodiments, patients treated by the methods disclosed herein have undergone a consolidation regimen and are in complete remission, and administering IL-2 and an agent as described herein post-consolidation regimen reduces the probability of occurrence of a relapsed or refractory AML. In some embodiments, patients treated by the methods disclosed herein have undergone a consolidation regimen and are in CR but have MRD, and administering IL-2 and an agent as described herein post-consolidation regimen reduces the probability of occurrence of relapsed or refractory AML. In some embodiments, patients treated by the methods disclosed herein have undergone a consolidation regimen and have MRD, and administering IL-2 and an agent as described herein post-consolidation regimen reduces the probability of occurrence of relapsed or refractory AML.

In some embodiments, the administration of IL-2 and the agent described in the methods herein is post-consolidation therapy or maintenance therapy. As used therein, the term “maintenance therapy” shall be given its ordinary meaning and shall also refer to an extended therapy, usually administered at a diminished dose that follows another treatment regimen (e.g., administration of IL-2 and an agent disclosed herein that follows one or more other forms of chemotherapy). In some embodiments, the maintenance therapy is administered to a patient who has one or more cancers in remission to reduce, delay or prevent a relapse or recurrence of the cancer(s) in the patient, and/or lengthening the time that the patient who has suffered from the cancer(s) remains in remission. Complete remission is not necessary for initiating maintenance therapy, as the maintenance therapy can be administered to a patient when a complete cure or remission is not attainable.

### NPM1 Mutants

In some embodiments, a patient treated by the methods disclosed herein has a mutant NPM1. In some embodiments, the mutant NPM1 comprises one or more mutations that cause cytoplasmic location of NPM1 protein. Various NPM1 mutations, methods of detecting NPM1 mutations, and compositions for detecting NPM1 mutations (e.g., antibodies specific for mutant NPM1, primers and/or probes for specifically amplifying and/or specifically detecting the presence of one or more NPM1 mutations in a patient nucleic acid) have been described in the art, including but not limited to U.S. Pat. Nos. 8,222,370, 8,501,924, 9,725,767, U.S. 2015/0368726, U.S. 2018/0119233, U.S. Pat. No. 8,877,910, U.S. 2010/0099084, and Bullinger, et al., New England Journal of Medicine 2004; 350:1605-1616, the entirety of each of which is hereby incorporated by reference.

In some embodiments, the mutant NPM1 comprises one or more of the NPM1 mutations depicted in Table 1 of U.S. Pat. No. 8,877,910, the entirety of which is hereby incorporated by reference. In some embodiments, the mutant NPM1 comprises one or more mutations in exon 12 NPM1 that cause cytoplasmic location of NPM1 protein. For example, the mutant NPM1 can comprise one or more of the NPM1 mutations selected from the group consisting of: Mutation A, Mutation B, Mutation C, Mutation D, Mutation E, Mutation F, Mutation E*, Mutation G*, Mutation H*, Mutation J, Mutation L, Mutation K, Mutation M, Mutation N, Mutation O, Mutation P, Mutation Q, Mutation Gm, Mutation Km, Mutation Lm, Mutation Nm, Mutation Om, Mutation Qm, Mutation 1, Mutation 3, Mutation 4, Mutation 6, Mutation 7, Mutation 12, Mutation 13, Mutation 10, Mutation 14, Mutation G+, Mutation H+, Mutation I+, Mutation J+, Mutation I, and a combination thereof. In some embodiments, the mutant NPM1 comprises one or more of the following NPM1 mutations: Mutation A, Mutation B, Mutation C, Mutation D, Mutation E or Mutation F. In some embodiments, the mutant NPM1 comprises a signal motif of nuclear export (NES) in exon 12 of NPM1. In some such embodiments, the NES comprises the amino acid sequence YxxxYxxYxY, wherein Y is a hydrophobic amino acid selected from the group consisting of leucine, isoleucine, methionine, valine, phenylalanine, and wherein x can be any amino acid.

In some embodiments, the presence of the mutant NPM1 is determined by identifying mutant NPM1 protein in patient cells. For example, in some embodiments, the mutant NPM1 protein is identified in the cells by identifying NPM1 protein in cytoplasm of the cells. In some such embodiments, the mutant NPM1 protein is identified in cytoplasm of the cells immunohistochemically. In some embodiments, the mutant NPM1 protein is identified in the cells with an antibody that selectively binds to the mutant NPM1 protein but not a wildtype NPM1 protein.

In some embodiments disclosed herein, the presence of the mutant NPM1 in a patient is determined by identifying a nucleic acid encoding the mutant NPM1 in the patient, for example a biological sample or a derivative thereof from the patient. In some embodiments, the nucleic acid is genomic DNA and/or mRNA. In some embodiments, the nucleic acid is obtained from an acellular body fluid (e.g., serum and/or plasma) of said patient. In some embodiments, identifying a nucleic acid encoding the mutant NPM1 comprises amplification of at least a portion of exon 12 of NPM1. In some embodiments, the amplification comprises polymerase chain reaction (PCR), such as, for example, real-time PCR (RT-PCR). In some embodiments, identifying a nucleic acid encoding the mutant NPM1 comprises using an oligonucleotide probe complimentary to a portion of exon 12 of NPM1. In some such embodiments, the oligonucleotide probe comprises a label (e.g., a fluorescent label). In some embodiments, the probe specifically hybridizes to either the wildtype NPM1 sequence or an NPM1 sequence comprising an insertion mutation.

Also disclosed herein include methods of acquiring knowledge of the presence of one or more molecular alterations in a biological sample from an AML patient. As used herein, the term “one or more molecular alterations” shall be given its ordinary meaning and shall also refer to any variation in the genetic or protein sequence in or more cells of a patient as compared to the corresponding wild-type genes or proteins. One or more molecular alterations include, but are not limited to, genetic mutations, gene amplifications, splice variants, deletions, insertions/deletions, gene rearrangements, single-nucleotide variations (SNVs), insertions, and aberrant RNA/protein expression. In some embodiments, said one or more molecular alterations comprises the presence of mutant NPM1. In some embodiments, knowledge of the presence of mutant NPM1 is acquired from an analytical assay, such as, for example, nucleic acid sequencing, polypeptide sequencing, restriction digestion, capillary electrophoresis, nucleic acid amplification-based assays, nucleic acid hybridization assay, comparative genomic hybridization, real-time PCR, quantitative reverse transcription PCR (qRT-PCR), PCR-RFLP assay, HPLC, mass-spectrometric genotyping, fluorescent in-situ hybridization (FISH), next generation sequencing (NGS), a kinase activity assay, and any combination thereof. In some embodiments, knowledge of the presence of mutant NPM1 is acquired from an antibody-based assay, such as, for example, ELISA, immunohistochemistry, western blotting, mass spectrometry, flow cytometry, protein-microarray, immunofluorescence, a multiplex detection assay, or any combination thereof. In some embodiments, knowledge of the presence of mutant NPM1 is acquired from immunohistochemistry.

In some embodiments, an electrophoretic mobility assay is used to acquire the knowledge of the one or more molecular alterations in the biological sample obtained from a patient. For example, a nucleic acid sequence encoding an NPM1 mutation is detected by amplifying the exon 12 of NPM1 and comparing the electrophoretic mobility of the amplified nucleic acid to the electrophoretic mobility of the corresponding region in a wild-type NPM1 gene.

In some embodiments, the analytical assay used to acquire the knowledge of the one or more molecular alterations in the biological sample involves polymerase chain reactions (PCR) or nucleic acid amplification-based assays. A number of PCR-based analytical assays known in the art are suitable for the methods disclosed herein, comprising but not limited to real-time PCR, quantitative reverse transcription PCR (qRT-PCR), and PCR-RFLP assay.

In some embodiments, the analytical assay used to acquire the knowledge of the one or more molecular alterations in the biological sample involves determining a nucleic acid sequence and/or an amino acid sequence comprising the one or more molecular alterations. In some embodiments, the nucleic acid sequence comprising the one or more molecular alterations from a cancer patient is sequenced. In some embodiments, the sequence is determined by a next generation sequencing procedure. As used herein “next-generation sequencing” refers to oligonucleotide sequencing technologies that have the capacity to sequence oligonucleotides at speeds above those possible with conventional sequencing methods (e.g. Sanger sequencing), due to performing and reading out thousands to millions of sequencing reactions in parallel. Non-limiting examples of next-generation sequencing methods/platforms include Massively Parallel Signature Sequencing (Lynx Therapeutics); solid-phase, reversible dye-terminator sequencing (Solexa/Illumina); DNA nanoball sequencing (Complete Genomics); SOLiD technology (Applied Biosystems); 454 pyro-sequencing (454 Life Sciences/Roche Diagnostics); ion semiconductor sequencing (ION Torrent); and technologies available from Pacific Biosciences, Intelligen Bio-systems, Oxford Nanopore Technologies, and Helicos Biosciences. Accordingly, in some embodiments, the NGS procedure used in the methods disclosed herein can comprise pyrosequencing, sequencing by synthesis, sequencing by ligation, or a combination of any thereof. In some embodiments, the NGS procedure is performed by an NGS platform selected from Illumina, Ion Torrent, Qiagen, Invitrogen, Applied Biosystem, Helicos, Oxford Nanopore, Pacific Biosciences, and Complete Genomics.

In some embodiments, the analytical assay used to acquire the knowledge of the one or more molecular alterations in the biological sample involves a nucleic acid hybridization assay that includes contacting nucleic acids derived from the biological sample with a nucleic acid probe comprising (1) a nucleic acid sequence complementary to a nucleic acid sequence encoding the NPM1 one or more mutations and further comprising (2) a detectable label. As used herein, the term “detectable label” shall be given its ordinary meaning and shall also refer to a molecule or a compound or a group of molecules (e.g., a detection system) used to identify a target molecule of interest. Typically, detectable labels represent a component of a detection system and are attached to another molecule that specifically binds to the target molecule. In some cases, the detectable label may be detected directly. In other cases, the detectable label may be a part of a binding pair, which can then be subsequently detected. Signals from the detectable label may be detected by various means and will depend on the nature of the detectable label. Examples of means to detect detectable label include but are not limited to spectroscopic, photochemical, biochemical, immunochemical, electromagnetic, radiochemical, or chemical means, such as fluorescence, chemifluorescence, chemiluminescence, or any other appropriate means.

In some embodiments, the biological sample comprises sputum, bronchoalveolar lavage, pleural effusion, tissue, whole blood, serum, plasma, buccal scrape, saliva, cerebrospinal fluid, urine, stool, circulating tumor cells, circulating nucleic acids, bone marrow, or any combination thereof. In some embodiments, the biological sample includes whole blood and blood components. In some embodiments, the blood component comprises plasma. In some embodiments, the biological sample is obtained from a patient before post-consolidation therapy. In some embodiments, the biological sample is obtained from a patient after a round of post-consolidation therapy. In some embodiments, the biological sample is obtained from a patient before induction therapy. In some embodiments, the biological sample is obtained from a patient after induction therapy. In some embodiments, the nucleic acid of the acellular fluid may be amplified in order to facilitate NPM1 mutation analysis. Methods of plasma and serum preparation are well known in the art.

### Methods of Treatment

Disclosed herein include methods of improving a survival rate of patients having acute myeloid leukemia (AML). In some embodiments, the method comprises (a) identifying the presence of mutant nucleophosmin 1 (NPM1) in a patient having AML; and (b) administering to a patient identified as having a mutant NPM1 in step (a) a therapeutically effective amount of IL2 and a therapeutically effective amount of an agent disclosed herein. In some embodiments, the method comprises (a) acquiring knowledge of the presence of one or more molecular alterations in a biological sample from an AML patient, wherein said one or more molecular alterations comprises the presence of mutant NPM1; and (b) for a patient known to have a mutant NPM1 in step (a), administering to the patient a therapeutically effective amount of IL2 and a therapeutically effective amount of an agent disclosed herein. In some embodiments, administration of IL-2 and the agent results in an increase in a survival rate of the treated patients compared to the untreated patients. In some embodiments, the administration of IL-2 and the agent results in an increase of at least about 10%, at least about 20%, at least about 30%, at least about 40%, at least about 50%, at least about 60%, at least about 65%, at least about 70%, at least about 80%, at least about 90%, at least about 100%, or higher, in the survival rate of the treated patients compared to the untreated patients. In some embodiments, the administration of IL-2 and the agent results in an increase of at least 30% in a survival rate of treated patients compared to the untreated patients. In some embodiments, the administration of IL-2 and the agent results in an increase of at least 30% in a survival rate of treated patients compared to the untreated patients. In some embodiments, the administration of IL-2 and the agent results in an increase of at least 50% in a survival rate of treated patients compared to the untreated patients.

In some embodiments, the survival rate is leukemia-free survival rate. In some embodiments, the survival rate is overall survival rate. Durations of leukemia-free survival (LFS) are measured as the time from random assignment of the patients to the date of relapse or death from any cause, whichever occurred first. Durations of overall survival (OS) are measured as the time from the date of random assignment to death from any cause. Durations of LFS and/or OS of the patients treated with combination treatment of IL-2 and the agent disclosed herein are compared with the duration of LFS and/or OS of the untreated patients. The average duration of survival of the patients treated with combination treatment of IL-2 and the agent disclosed herein is compared with the average duration of survival of the untreated patients. In some embodiments, the survival rate of the patient treated with combination treatment of IL-2 and the agent disclosed herein is compared with the survival rate of the untreated patients. In some embodiments, the Kaplan-Meier procedure is used to estimate the survival distributions and survival rate for a population of patients. In some embodiments, the administration of IL-2 and the agent results in an increase of the patient's LFS and/or OS time by at least 1.1 fold (e.g., 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, or any overlapping ranges) or more relative to the duration of LFS and/or OS of the untreated patients.

Further disclosed herein include methods of preventing and/or delaying the onset of relapse to AML in a patient in complete remission (CR) from AML. In some embodiments, the method comprises the steps of: (a) identifying the presence of mutant NPM1 in a patient in CR from AML; and (b) administering to a patient identified as having a mutant NPM1 in step (a) a therapeutically effective amount of IL2 and a therapeutically effective amount of an agent selected from the group consisting of histamine, a histamine structural analog having H₂-receptor activities, an endogenous histamine releasing preparation, a non-histamine derivative H₂-receptor agonist, and a combination thereof. In some embodiments, the method comprises (a) acquiring knowledge of the presence of one or more molecular alterations in a biological sample from an AML patient, wherein said one or more molecular alterations comprises the presence of mutant NPM1; and (b) for a patient known to have a mutant NPM1 in step (a), administering to the patient a therapeutically effective amount of IL2 and a therapeutically effective amount of an agent selected from the group consisting of histamine, a histamine structural analog having H2-receptor activities, an endogenous histamine releasing preparation, a non-histamine derivative H₂-receptor agonist, and a combination thereof. In some embodiments, the administration of IL-2 and the agent prevents and/or delays the onset of relapse to AML in said patient. In some embodiments, relapse comprises at least 1% (e.g., 1%, 2%, 3%, 4%, 5%, 6%, 7%, 8%, 9%, 10%, 12%, 15%, 20%, and overlapping ranges) blast cells in the bone marrow. In some embodiments, relapse comprises extramedullary leukemia. In some embodiments, the administration of IL-2 and the agent delays relapse of AML of treated patients by at least 1 week (e.g., 7 days, 10 days, 30 days, 2 months, 3 months, 4 months, 5 months, 6 months, 7 months, 8 months, 9 months, 10 months, 11 months, 1 year, 18 months, 2 years, 30 months, 3 years, 40 months, 4 years, 5 years, 6 years, 7 years, 8 years, 9 years, 10 years, 15 years, 20 years, 25 years, 30 years, 35 years, 40 years, 50 years, 55 years, 60 years, 65 years, 70 years, 75 years, and overlapping ranges) compared to the untreated patients. In some embodiments, the administration of IL-2 and the agent delays relapse of AML of treated patients by at least 3 months compared to the untreated patients. In some embodiments, the administration of IL-2 and the agent delays relapse of AML of treated patients by at least 6 months compared to the untreated patients. In some embodiments, the administration of IL-2 and the agent delays relapse of AML of treated patients by at least 12 months compared to the untreated patients.

Further disclosed herein include methods of prolonging remission from AML, comprising the steps of: (a) identifying the presence of mutant NPM1 in a patient in remission from AML; and (b) administering to a patient identified as having a mutant NPM1 in step (a) a therapeutically effective amount of IL2 and a therapeutically effective amount of an agent selected from the group consisting of histamine, a histamine structural analog having H₂-receptor activities, an endogenous histamine releasing preparation, a non-histamine derivative H₂-receptor agonist, and a combination thereof. In some embodiments, the method comprises (a) acquiring knowledge of the presence of one or more molecular alterations in a biological sample from an AML patient, wherein said one or more molecular alterations comprises the presence of mutant NPM1; and (b) for a patient known to have a mutant NPM1 in step (a), administering to the patient a therapeutically effective amount of IL2 and a therapeutically effective amount of an agent selected from the group consisting of histamine, a histamine structural analog having H₂-receptor activities, an endogenous histamine releasing preparation, a non-histamine derivative H₂-receptor agonist, and a combination thereof. In some embodiments, the administration of IL-2 and the agent prolongs remission from AML in said patient. In some embodiments, the administration of IL-2 and the agent prolongs remission from AML of the treated patients by at least 1 week (e.g., 7 days, 10 days, 30 days, 2 months, 3 months, 4 months, 5 months, 6 months, 7 months, 8 months, 9 months, 10 months, 11 months, 1 year, 18 months, 2 years, 30 months, 3 years, 40 months, 4 years, 5 years, 6 years, 7 years, 8 years, 9 years, 10 years, 15 years, 20 years, 25 years, 30 years, 35 years, 40 years, 50 years, 55 years, 60 years, 65 years, 70 years, 75 years, and overlapping ranges) compared to the untreated patients. In some embodiments, the administration of IL-2 and the agent prolongs remission from AML of the treated patients by at least 3 months compared to the untreated patients. In some embodiments, the administration of IL-2 and the agent prolongs remission from AML of treated patients by at least 6 months compared to the untreated patients. In some embodiments, administration of IL-2 and the agent prolongs remission from AML of treated patients by at least 12 months compared to the untreated patients.

It should be appreciated by those of skill in the art that in some embodiments the compositions and methods described herein preferably selectively affect leukemic cells without affecting normal cells (e.g., leukocytes) in the population of cells. In some embodiments, leukemic cells are selectively eradicated without eradicating normal leukocytes in the population of cells. For example, the leukemic cells are selectively eradicated without eradicating normal bone marrow leukocytes or normal peripheral blood leukocytes, including without limitation, stem and progenitors, bone marrow mononuclear cells, myeloblasts, neutrophils, NK cells, macrophages, granulocytes, monocytes, and lineage−/cKit+/Scal+(LKS) cells. In some embodiments, the amount or activity of leukemic cells in a population of cells is selectively decreased without decreasing the amount or activity of normal leukocytes in the population. In some embodiments, proliferation of leukemic cells is selectively inhibited in a population of cells without inhibiting proliferation of normal leukocytes in the population, In some embodiments, the compositions and methods described herein can be used to increase the number of normal leukocytes in a population of cells by selectively reducing the number, activity, and/or proliferation of leukemic cells in the population of cells. Without wishing to be bound by theory, it is expected that the amount of leukemic cells eradicated, reduced, or inhibited in any particular population of cells is proportional to the concentration of IL-2 and the agent disclosed herein to which the population of cells has been exposed. In some instances, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or as much as 100% of the leukemic cells in the population of cells are eradicated, reduced, or inhibited by exposure to or contact with IL-2 and the agent disclosed herein. In some embodiments, at least 20% of the leukemic cells in the population of cells are eradicated, reduced, or inhibited. In some embodiments, at least 50% of the leukemic cells in the population of cells are eradicated, reduced, or inhibited. In some embodiments, at least 70% of the leukemic cells in the population of cells are eradicated, reduced, or inhibited. In some embodiments, all of the leukemic cells in the population of cells are eradicated, reduced, or inhibited.

In some of the methods disclosed herein, the IL-2 and agent disclosed herein can be administered in combination with another acute myeloid leukemia therapy, such as, for example, chemotherapy, stem cell transplantation therapy, a hypomethylating agent therapy, a FLT3 inhibitor therapy, a farnesyltransferase inhibitor therapy, a topoisomerase II inhibitor therapy, a P-glycoprotein modulator therapy, or a combinations thereof.

In some embodiments, the chemotherapeutic agent is a cell cycle inhibitor. As used herein the term “cell cycle inhibitor” shall be given its ordinary meaning and shall also refer to a chemotherapeutic agent that inhibits or prevents the division and/or replication of cells. In some embodiments, the term “cell cycle inhibitor” includes a chemotherapeutic agent selected from Doxorubicin, Melphlan, Roscovitine, Mitomycin C, Hydroxyurea, 50Fluorouracil, Cisplatin, Ara-C, Etoposide, Gemcitabine, Bortezomib, Sunitinib, Sorafenib, Sodium Valproate, HDAC Inhibitors, or Dacarbazine. Examples of HDAC inhibitors include, but are not limited to, FR01228, Trichostatin A, SAHA and PDX101. In some embodiments, the cell cycle inhibitor is a DNA synthesis inhibitor. As used herein the term “DNA synthesis inhibitor” shall be given its ordinary meaning and shall also refer to a chemotherapeutic agent that inhibits or prevents the synthesis of DNA by a cancer cell. Examples of DNA synthesis inhibitors include, but are not limited to, AraC (cytarabine), 6-mercaptopurine, 6-thioguanine, 5-fluorouracil, capecitabine, floxuridine, gemcitabine, decitabine, vidaza, fludarabine, nelarabine, cladribine, clofarabine, pentostatin, thiarabine, troxacitabine, sapacitabine or forodesine. In some embodiments, the DNA synthesis inhibitor is cytarabine or another deoxycytidine analogue as described herein. In some embodiments, the DNA synthesis inhibitor is a DNA elongation terminator and functions in a similar way to cytarabine such as fludarabine, nelarabine, cladribine, or clofarabine. As used herein, “AraC” (Arabinofuranosyl Cytidine) shall be given its ordinary meaning and shall also refer to a compound comprising a cytosine base and an arabinose sugar that is converted into Arabinofuranosylcytosine triphosphate in vivo. AraC is also known as cytarabine or cytosine arabinoside. FLT3 inhibitors include, but are not limited to, Semexanib (SU5416), Sunitinib (SU11248), Midostaurin (PKC412), Lestautinib (CEP-701), Tandutinib (MLN518), CHIR-258, Sorafenib (BAY-43-9006) and KW-2449. Farnesyltransferase inhibitors include, but are not limited to, tipifarnib (R115777, Zarnestra), lonafarnib (SCH66336, Sarasar™) and BMS-214662. Topoisomerase II inhibitors include, but are not limited to, the epipodophyllotoxins etoposide and teniposide, and the anthracyclines doxorubicin and 4-epi-doxorubicin. P-glycoprotein modulators include, but are not limited to, zosuquidar trihydrochloride (Z.3HCL), vanadate, and/or verapamil. Hypomethylating agents include, but are not limited to, 5-aza-cytidine and/or 2′ deoxyazacitidine.

In some embodiments, the IL-2 and the agent disclosed herein and the chemotherapeutic agent are administered to the patient at the same time, optionally as a composition comprising the IL-2 and the agent disclosed herein and the chemotherapeutic agent, or as two separate doses. In some embodiments, the IL-2 and the agent disclosed herein and the chemotherapeutic agent are used or administered to the patient at different times. For example, in some embodiments, the IL-2 and the agent disclosed herein are administered prior to, or after the chemotherapeutic agent. In some embodiments, the IL-2 and the agent disclosed herein are administered prior to, or after the chemotherapeutic agent separated by a time of at least 1 minute, 2 minutes, 5 minutes, 10 minutes, 30 minutes, 45 minutes, 1 hour, 1.5 hours, 2 hours, 3 hours, 4 hours, 5 hours, 8 hours, 10 hours, 12 hours 16 hours, or 24 hours. Optionally, in some embodiments the IL-2 and the agent disclosed herein and chemotherapeutic agent are administered to the patient separated by more than 24 hours, 36 hours, 48 hours, 3 days, 4 days, 5 days, 6 days, one week, 10 days, 12 days, two weeks, three weeks, one month, 6 weeks, 2 months, or greater than 2 months. In some embodiments, the IL-2 and the agent disclosed herein are administered or used between 2 days and 7 days after the chemotherapeutic agent.

As noted above, other therapeutic regimens may be combined with the administration of IL-2 and the agent disclosed herein. The combined administration includes co-administration, using separate formulations or a single pharmaceutical formulation, and consecutive administration in either order, wherein preferably there is a time period while both (or all) active agents simultaneously exert their biological activities. Preferably such combined therapy results in a synergistic therapeutic effect.

In some embodiments, the methods provided herein i) improve a survival rate, ii) delay and/or prevent the onset of relapse, and/or iii) prolong remission from a cancer other than AML. In some embodiments, a patient treated by the methods disclosed herein has or is suffering from AML. In some embodiments, a patients treated by the methods disclosed herein is in remission from a cancer other than AML. In some such embodiments, the cancer is NPM1 mutated. In some embodiments, the cancer is a leukemia. In some embodiments, the leukemia is Chronic myeloid leukemia (CML). In some embodiments, the leukemia is Chronic myelomonocytic leukemia (CMML). In some embodiments, the leukemia is Acute lymphocytic leukemia (ALL). In some embodiments, the leukemia is Chronic lymphocytic leukemia (CLL). In some embodiments, the leukemia is hairy cell leukemia. In some embodiments, the patient has or has had a tumor. In some embodiments, the tumor is a solid tumor, such as, for example, a colon carcinoma, prostate cancer, breast cancer, lung cancer, skin cancer, liver cancer, bone cancer, ovary cancer, pancreas cancer, brain cancer, head and neck cancer. In some embodiments, the cancer or tumor is in the breast, prostate, lung, colon, stomach, pancreas, ovary, and/or brain. In some embodiments, the cancer is a hematopoietic cancer, a neuroblastoma, or a malignant glioma. In some embodiments, the cancer is selected from one or more of the following: Adrenocortical Carcinoma, AIDS-Related Cancers, Kaposi Sarcoma, AIDS- Related Lymphoma, Primary CNS Lymphoma, Anal Cancer, Appendix Cancer, Astrocytomas, Atypical Teratoid/Rhabdoid Tumor, Central Nervous System, Basal Cell Carcinoma - see Skin Cancer (Nonmelanoma), Bile Duct Cancer, Bladder Cancer, Bone Cancer, Ewing Sarcoma Family of Tumors, Osteosarcoma and Malignant Fibrous Histiocytoma, Brain Stem Glioma, Brain Tumor, Astrocytomas, Brain and Spinal Cord Tumors, Brain Stem Glioma, Central Nervous System Atypical Teratoid/Rhabdoid Tumor, Central Nervous System Embryonal Tumors, Central Nervous System Germ Cell Tumors, Craniopharyngioma, Ependymoma, Breast Cancer, Bronchial Tumors, Burkitt Lymphoma—see Non-Hodgkin Lymphoma, Carcinoid Tumor, Gastrointestinal, Carcinoma of Unknown Primary, Cardiac (Heart) Tumors, Central Nervous System, Atypical Teratoid/Rhabdoid Tumor, Embryonal Tumors, Germ Cell Tumor, Lymphoma, Primary, Cervical Cancer, Cholangiocarcinoma, Chordoma, Chronic Myeloproliferative Neoplasms, Colon Cancer, Colorectal Cancer, Craniopharyngioma, Cutaneous T-Cell Lymphoma - see Mycosis Fungoides and Sezary Syndrome, Ductal Carcinoma In Situ (DCIS), Embryonal Tumors, Central Nervous System, Endometrial Cancer, Ependymoma, Esophageal Cancer, Esthesioneuroblastoma, Ewing Sarcoma, Extracranial Germ Cell Tumor, Extragonadal Germ Cell Tumor, Eye Cancer, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST), Germ Cell Tumor, Central Nervous System, Extracranial, Extragonadal, Ovarian, Testicular, Gestational Trophoblastic Disease, Glioma - see Brain Tumor Brain Stem, Head and Neck Cancer, Heart Cancer, Hepatocellular (Liver) Cancer, Histiocytosis, Langerhans Cell, Hodgkin Lymphoma, Hypopharyngeal Cancer, Intraocular Melanoma, Islet Cell Tumors, Pancreatic Neuroendocrine Tumors, Kaposi Sarcoma, Kidney, Renal Cell, Wilms Tumor and Other Childhood Kidney Tumors, Langerhans Cell Histiocytosis, Laryngeal Cancer, Hairy Cell, Lip and Oral Cavity Cancer, Liver Cancer (Primary), Lung Cancer, Non-Small Cell, Small Cell, Lymphoma, AIDS-Related, Burkitt—see Non-Hodgkin Lymphoma, Cutaneous T-Cell—see Mycosis Fungoides and Sezary Syndrome, Hodgkin, Non- Hodgkin, Primary Central Nervous System (CNS), Macroglobulinemia, Waldenstrom—see Non-Hodgkin Lymphoma, Male Breast Cancer, Malignant Fibrous Histiocytoma of Bone and Osteosarcoma, Melanoma, Intraocular (Eye), Merkel Cell Carcinoma, Mesothelioma, Malignant, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myeloma, Multiple, Myeloproliferative Neoplasms, Chronic, Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip and, Oropharyngeal Cancer, Osteosarcoma and Malignant Fibrous Histiocytoma of Bone, Ovarian Cancer, Epithelial, Germ Cell Tumor, Low Malignant Potential Tumor, Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma, Ewing, Kaposi, Osteosarcoma (Bone Cancer), Rhabdomyosarcoma, Soft Tissue, Uterine, Vascular Tumors, Sezary Syndrome, Skin Cancer, Melanoma, Merkel Cell Carcinoma, Nonmelanoma, Small Cell Lung Cancer, Small Intestine Cancer, Soft Tissue Sarcoma, Squamous Cell Carcinoma—see Skin Cancer (Nonmelanoma), Squamous Neck Cancer with Occult Primary, Metastatic, Stomach (Gastric) Cancer, T-Cell Lymphoma, Cutaneous—see Mycosis Fungoides and Sezary Syndrome, Testicular Cancer, Throat Cancer, Thymoma and Thymic Carcinoma, Thyroid Cancer, Transitional Cell Cancer of the Renal Pelvis and Ureter, Primary Carcinoma, Ureter and Renal Pelvis, Transitional Cell Cancer, Urethral Cancer, Uterine Cancer, Endometrial, Uterine Sarcoma, Vaginal Cancer, Vascular Tumors, Vulvar Cancer, Waldenstrom Macroglobulinemia, Non- Hodgkin Lymphoma, and Wilms Tumor.

### Combination Therapy

In some embodiments, the methods disclosed herein comprise administering to a patient having a mutant NPM1 a therapeutically effective amount of IL-2 and a therapeutically effective amount of an agent disclosed herein (for example, histamine, a histamine structural analog having H₂-receptor activities, an endogenous histamine releasing preparation, a non-histamine derivative H₂-receptor agonist, or a combination thereof). In some embodiments, the methods disclosed herein comprise administering to a patient having a mutant NPM1 a therapeutically effective amount of a cytokine and a therapeutically effective amount of an agent disclosed herein. In some embodiments, a cytokine other than IL-2 is administered. In some embodiments, the cytokine is an interleukin, such as, IL-2, IL-12, and/or IL-15. In some embodiments, the cytokine is an interferon, such as, for example, interferon-alpha, interferon-beta, and/or interferon-gamma In some embodiments, the cytokine is a hematopoietic growth factor, for example, Erythropoietin, IL-11, Granulocyte-macrophage colony-stimulating factor (GM-CSF), and/or granulocyte colony-stimulating factor (G-CSF).

The agent comprises, in some of embodiments of the methods disclosed herein, one or more of histamine, histamine salts, histamine prodrugs, histamine receptor agonists, histamine esters, histamine structural analogs, endogenous histamine-releasing preparations, and non-histamine derivative H₂-receptor agonists.

In some embodiments, the agent is histamine. In some embodiments, the histamine is histamine dihydrochloride. In some embodiments, the histamine is N-methyl-histamine. In some embodiments, the N-methyl-histamine comprises Na-methyl-histamine dihydrochloride (NMH). In some embodiments, the histamine is 4-methyl-histamine. Histamine dihydrochloride is commercially available and methods of making histamine dihydrochloride as well as other forms of histamine are known in the art (e.g., U.S. Pat. No. 6,528,654, which is incorporated herein by reference in its entirety). The agent comprises, in some of embodiments of the methods disclosed herein, one or more of histamine salts, histamine esters, and/or histamine prodrugs. Histamine can, for example, suppress a variety of immune effector mechanisms in vitro. This property of histamine is H₂-receptor associated. Examples of histamine salts include, but are not limited to, histamine dihydrochloride (HDC, e.g., HDC sold under the tradename of Ceplene®), histamine phosphate and histamine diphosphate. Non-limiting examples of histamine esters and histamine prodrugs are described in U.S. Pat. No. 6,613,788, which is hereby incorporated by reference in its entirety.

As used herein, the term “H₂-receptor agonist” shall be given its ordinary meaning and shall also refer to a compound, such as histamine, that is capable of binding to histamine H₂-receptor on the surface of a cell and triggers the transduction of a signal over the cell membrane. The term H₂-receptor agonist includes agonist compounds that are structurally similar to histamine (i.e., histamine analogs) as well as agonists that are structurally unrelated to histamine. Analogs of histamine having H₂-receptor activities which are suitable for use in the present application are known in the art, for example, 4-methyl histamine. By means of example, the analogs can have a chemical structure similar to that of histamine but be modified by the addition of moieties which do not negatively interfere with their histamine-like activities, and in particular with their H₂-receptor agonist activities. Non-limiting examples of non-histamine derivative H₂-receptor agonists suitable for use herein are those such as dimaprit but not N-methyl-dimaprit or nor-dimaprit. This pharmacological terminology is explained in more detail in “Chemistry and Structure-Activity Relationships of Drugs Acting as Histamine Receptors,” Pharmacology of Histamine Receptors, Ganellin et al, John Wright & Sons, Bristol, pages 10-102 (1982).

As used herein, compounds referred to as “endogenous histamine-releasing preparation” shall be given their ordinary meaning and shall also refer to compounds which cause the level of histamine in a patient to increase either by increasing histamine's production/release or by inhibiting histamine breakdown/elimination to increase levels of histamine in a patient as more is released. This is an alternative to directly treating with histamine Endogenous histamine releasing preparations suitable for use herein are known in the art. Examples of preparations capable of releasing endogenous histamine include, but are not limited to, compounds comprising other lymphokines such as IL-3 or allergens. However, other known preparations are also suitable. For example, compounds which liberate intracellular stores of histamine either into the circulation of a patient or into the tissue of cells adjacent to histamine-containing cells are also encompassed by the phrase “endogenous histamine-releasing preparation. The administration of compounds which increases the level of histamine in a patient induces effects similar to those noted after the administration of histamine. Examples of histamine releasing drugs are listed in “Factors Regulating Availability of Histamine at Tissue Receptors,” Pharmacology of Histamine Receptors, Ganellin et al, John Wright & Sons, Bristol, pages 103-145 (1982), hereby incorporated by reference in its entirety.

In some embodiments, the methods and uses described herein involve the administration or use of an effective amount of IL-2 and an agent disclosed herein. As used herein, the terms “effective amount” and “therapeutically effective amount” shall be given their ordinary meanings and shall also refer to an amount effective, at dosages and for periods of time necessary to achieve the desired result. For example in the context of improving a survival rate of patients having acute myeloid leukemia (AML), an effective amount is an amount that for example prolongs remission, reduces switching from MRD negative to MRD positive, and/or prevents tumor spread or growth of leukemic cells compared to the response obtained without administration of the compounds. Effective amounts may vary according to factors such as the disease state, age, sex and weight of the animal. The amount of a given compound that will correspond to such an amount will vary depending upon various factors, such as the given drug or compound, the pharmaceutical formulation, the route of administration, the type of disease or disorder, the identity of the patient or host being treated, and the like, but can nevertheless be routinely determined by one skilled in the art.

As disclosed herein, co-administration of particular ratios and/or amounts of IL-2 and the agent disclosed herein can result in synergistic effects in i) improving a survival rate, ii) delaying and/or preventing relapse to AML, and/or iii) prolonging remission from AML. These synergistic effects can be such that the one or more effects of the combination compositions are greater than the one or more effects of each component alone at a comparable dosing level, or they can be greater than the predicted sum of the effects of all of the components at a comparable dosing level, assuming that each component acts independently. The synergistic effect can be about, or greater than about, 5%, 10%, 20%, 30%, 50%, 75%, 100%, 110%, 120%, 150%, 200%, 250%, 350%, or 500% better than the effect of treating a patient with one of the components alone, or the additive effects of each of the components when administered individually. The effect can be any of the measurable effects described herein. The composition comprising a plurality of components can be such that the synergistic effect is an enhancement in a survival rate and that survival rate is increased to a greater degree as compared to the sum of the effects of administering each component, determined as if each component exerted its effect independently, also referred to as the predicted additive effect herein. For example, if a composition comprising component (a) yields an effect of a 20% improvement in leukemia-free survival and a composition comprising component (b) yields an effect of 50% improvement in leukemia-free survival, then a combination composition comprising both component (a) and component (b) can be considered to have a synergistic effect if the combination composition's effect on leukemia-free survival was greater than 70%, 80%, 90%, 95%, 98%, or 99%. For example, the component (a) can be an agent disclosed herein, for example an agent selected from the group consisting of histamine, a histamine structural analog having H₂-receptor activities, an endogenous histamine releasing preparation, a non-histamine derivative H₂-receptor agonist, and a combination thereof. In some embodiments, the component (a) is histamine, for example histamine dihydrochloride. In some embodiments, the component (b) is an IL-2.

A synergistic combination composition can have an effect that is greater than the predicted additive effect of administering each component of the combination composition alone as if each component exerted its effect independently. For example, if the predicted additive effect is 70%, an actual effect of 140% is 70% greater than the predicted additive effect or is 1 fold greater than the predicted additive effect. The synergistic effect can be at least about 20%, 50%, 75%, 90%, 100%, 150%, 200% or 300% greater than the predicted additive effect. In some embodiments, the synergistic effect can be at least about 0.2, 0.5, 0.9, 1.1, 1.5, 1.7, 2, or 3 fold greater than the predicted additive effect.

In some embodiments, the synergistic effect of the combination compositions can also allow for reduced dosing amounts, leading to reduced side effects to the patient and reduced cost of treatment. Furthermore, the synergistic effect can allow for results that are not achievable through any other treatments. Therefore, proper identification, specification, and use of combination compositions can allow for significant improvements in i) improving a survival rate, ii) delaying and/or preventing relapse to AML, and/or iii) prolonging remission from AML.

In some embodiments, the therapeutic agents as described herein, comprising IL-2 and the agent disclosed herein, are administered once a day. In some embodiments, therapeutic agents are administered to a patient two times per day (BID). In some embodiments, therapeutic agents are administered to a patient three times per day. In some embodiments, therapeutic agents are administered to a patient four times per day. In some embodiments, therapeutic agents are administered to a patient once a week. In some embodiments, therapeutic agents are administered to a patient two times per week. In some embodiments, therapeutic agents are administered to a patient three times per week. In some embodiments, therapeutic agents are administered to a patient four times per week. In some embodiments, therapeutic agents are administered to a patient once every two weeks.

In some embodiments, any of the therapeutic or prophylactic drugs or compounds described herein may be administered simultaneously. In some embodiments, IL-2 and the agent disclosed herein are administered at different time than one another. In some embodiments, IL-2 and the agent disclosed herein are administered within a few minutes of one another. In some embodiments, IL-2 and the agent disclosed herein are administered within a few hours of one another. In some embodiments, IL-2 and the agent disclosed herein are administered within 1 hour of one another. In some embodiments, IL-2 and the agent disclosed herein are administered within 2 hours of one another. In some embodiments, IL-2 and the agent disclosed herein are administered within 5 hours of one another. In some embodiments, IL-2 and the agent disclosed herein are administered within 12 hours of one another. In some embodiments, IL-2 and the agent disclosed herein are administered within 24 hours of one another.

In some embodiments, the administration of IL-2 and an agent disclosed herein commences immediately after a cancer therapy (e.g., a primary cancer therapy one or more therapeutic agents, radiation therapy and/or surgery) has ceased. In some embodiments, the administration of IL-2 and an agent disclosed herein commences after a gap in time (e.g., 1, 5, 10, 15, 20, 25, 30 days; 1, 2, 4, 6, 8, 12 months; or 1, 1.5, 2, 2.5, 3, 5 years or longer) between the end of cancer therapy and the administration of IL-2 and an agent disclosed herein. In some embodiments, administration of IL-2 and an agent disclosed herein can continue for as long as relapse-free survival is maintained (e.g., up to about a day, a week, a month, six months, a year, two years, three years, four years, five years, or longer). In some embodiments, the IL-2 and agent disclosed herein are administered in a pre-determined schedule (e.g., continuous therapy followed by one or more of: drug free intervals, combinations with other cancer therapies, or alternating with other cancer therapies).

In some embodiments, the patient has completed induction chemotherapy. In some embodiments, the patient is a patient who relapses from complete remission of AML after induction chemotherapy. In some embodiments, the patient has completed induction and consolidation chemotherapy. In some embodiments, administration of IL-2 and the agent begins the same day after consolidation chemotherapy is completed. In some embodiments, administration of IL-2 and the agent begins between about 1 day (e.g., 24 hours, 36 hours, 48 hours, 3 days, 4 days, 5 days, 6 days, one week, 10 days, 12 days, two weeks, three weeks, one month, 6 weeks, 2 months, 4 months, 6 months, 8 months, 10 months, 12 months, 14 months, and ranges in-between) after consolidation chemotherapy is completed. In some embodiments, administration of IL-2 and the agent begins between about 300 days after consolidation chemotherapy is completed. In some embodiments, administration of IL-2 and the agent begins about 200 days after consolidation chemotherapy is completed. In some embodiments, administration of IL-2 and the agent begins about 100 days after consolidation chemotherapy is completed. In some embodiments, administration of IL-2 and the agent begins about 50 days after consolidation chemotherapy is completed.

As will be readily apparent to one of skill in the art, the useful in vivo dosage to be administered and the particular mode of administration can vary depending upon the age and weight of the patient, as well as the severity of the condition. The agent can be administered in amounts that an artisan with skill in the art can determine In some embodiments, IL-2 and the agent are administered in repeated 3-week cycles for about 3 months, about 6 months, about 9 months, about 12 months, about 18 months, about 2 years, about 3 years, or longer. The 3-week cycles of treatment can be separated by rest period of about 2 weeks, about 3 weeks, about 4 weeks, about 5 weeks, about 6 weeks, about 7 weeks, about 8 weeks, about 9 weeks or longer. In some embodiments, the administration of IL-2 and the agent coincides with the period of the highest risk of relapse in AML. In some embodiments, the agent is administered in an amount of about 0.1 mg/day to about 10 mg/day (e.g., 0.1 mg/day, 0.2 mg/day, 0.4 mg/day, 0.6 mg/day, 0.8 mg/day, 1.0 mg/day, 1.5 mg/day, 2.0 mg/day, 2.5 mg/day, 3.0 mg/day, 3.5 mg/day, 4.0 mg/day, 4.5 mg/day, 5.0 mg/day, 5.5 mg/day, 6.0 mg/day, 6.5 mg/day, 7.0 mg/day, 7.5 mg/day, 8.0 mg/day, 8.5 mg/day, 9.0 mg/day, 9.5 mg/day, 10.0 mg/day, or any of the overlapping range), more preferably about 0.5 mg/day to about 8 mg/day, and more preferably about 1 mg/day to about 5 mg/day for a period of time of about 1 week to about 1 month, and in some instances for a period greater than about 2 months, for example about 3 months, about 6 months, about 9 months, about 12 months, about 18 months, about 2 years, or about 3 years. In some embodiments, the IL-2 can be administered in an amount of about 1,000 U/kg/day to about 300,000 U/kg/day (e.g, 1,000 U/kg/day, 2,000 U/kg/day, 4,000 U/kg/day, 6,000 U/kg/day, 8,000 U/kg/day, 10,000 U/kg/day, 15,000 U/kg/day, 25,000 U/kg/day, 50,000 U/kg/day, 100,000 U/kg/day, 200,000 U/kg/day, 300,000 U/kg/day, and overlapping ranges), more preferably about 3,000 U/kg/day to about 100,000 U/kg/day, and more preferably about 5,000 U/kg/day to about 20,000 U/kg/day, for a period of about 1 week to about 1 month, and in some cases the treatment may be prolonged for a period greater than about 2 months. The treatment with the two compounds may be discontinued for a period of time and then resumed as was described above. Other regimes and amounts can also be utilized.

In some embodiments, the method comprises administrating the agent and IL-2 are once per day. In some embodiments the agent and IL-2 are administered for at least one (e.g., 1, 2, 3, 4, 5, 6, 7, 8, 10, 12, 14, 16, 18, and ranges in between) cycle. In some embodiments, the agent and IL-2 are administered for at least two cycles. In some embodiments, the agent and IL-2 are administered for at least six cycles. In some embodiments, one cycle comprises at least 2 (for example, 2, 3, 4, 5, 6, 7, 8, 10, 12, 14, 16, 18, 20, 22, 24, 26, 28, 30, 35, 40, and ranges in between) consecutive days of treatment. In some embodiments, one cycle comprises 21 consecutive days of treatment. In some embodiments, an interval between two treatment cycles is at least two (for example, 2, 3, 4, 5, 6, 7, 8, 10, 12, 14, 16, 18, 20, 22, 24, 26, 28, 30, 35, 40, and ranges in between) days. In some embodiments, an interval between two treatment cycles is at least two weeks. In some embodiments, an interval between two treatment cycles is at least three weeks. In some embodiments, an interval between two treatment cycles is at least six weeks.

In some embodiments, the method comprises administering the agent twice a day. In some embodiments, the agent is histamine. In some embodiments, the agent is histamine dihydrochloride. In some embodiments, the agent is histamine diphosphate. In some embodiments, the agent (e.g., histamine) is administered at 0.5 mg twice a day. In some embodiments, the method comprises administering IL-2 twice a day. In some embodiments, IL-2 is administered in an amount of about 5,000 U/kg/day to about 300,000 U/kg/day. In some embodiments, IL-2 is administered at a dosage of 16,400 U/kg twice a day.

In some embodiments, the administration of IL-2 and the agent disclosed herein may occur either simultaneously or time-staggered, either at the same site of administration or at different sites of administration. In some embodiments, the administration of the agent and/or IL-2 is accomplished by one or more of intramuscular injection, subcutaneous injection, intradermal injection, intravenous injection, implantation infusion device, inhalation, and transdermal diffusion. In some embodiments, the administration of the agent and/or IL-2 is accomplished by subcutaneous injection.

### Pharmaceutical Compositions and Formulations

Some embodiments of the methods disclosed herein relate methods of administering compositions, including pharmaceutical compositions, which include a therapeutically effective amount of IL-2 and the agent disclosed herein. In some embodiments, the compositions can include the IL-2 and/or the agent described herein and a pharmaceutically acceptable excipient and/or carrier. As used herein, the terms “physiologically acceptable” and “pharmaceutically acceptable” shall be given its ordinary meaning and shall also refer to a carrier, diluent or excipient that does not abrogate the biological activity and properties of IL-2 and the agent disclosed herein. As used herein, “pharmaceutical composition” shall be given its ordinary meaning and shall also refer to a therapeutically effective amount of IL-2 and/or an agent disclosed herein, together with a pharmaceutically acceptable carrier or diluent The pharmaceutical compositions can, in some embodiments, administered to a patient by any method known to a person skilled in the art, such as, for example, parenterally, transmucosally, transdermally, intramuscularly, intravenously, intra-dermally, intra-peritonealy, intra-ventricularly, intra-cranially, intra-vaginally or intra-tumorally. In some embodiments, the pharmaceutical composition is administered subcutaneously. IL-2 and agents described herein may also be administered by the intraperitoneal and other parenteral routes. Solutions of the active compound as a free acid or a pharmaceutically-acceptable salt may be administered in water with or without a surfactant such as hydroxypropyl cellulose. Dispersions are also contemplated such as those utilizing glycerol, liquid polyethylene glycols and mixtures thereof and oils. Antimicrobial compounds may also be added to the preparations. Injectable preparations may include sterile aqueous solutions or dispersions and powders which may be diluted or suspended in a sterile environment prior to use. Carriers such as solvents dispersion media containing, e.g., water, ethanol polyols, vegetable oils and the like, may also be added. Coatings such as lecithin and surfactants may be utilized to maintain the proper fluidity of the composition. Isotonic agents such as sugars or sodium chloride may also be added as well as products intended for the delay of absorption of the active compounds such as aluminum monostearate and gelatin. Sterile injectable solutions are prepared as is known in the art and filtered prior to storage and/or administration. Sterile powders may be vacuum dried freeze dried from a solution or suspension containing them. In some embodiments, the pharmaceutical compositions are administered by intravenous, intra-arterial, or intra-muscular injection of a liquid preparation. Suitable liquid formulations include solutions, suspensions, dispersions, emulsions, oils and the like. In some embodiments, the pharmaceutical compositions are administered intravenously and are thus formulated in a form suitable for intravenous administration. In some embodiments, the pharmaceutical compositions are administered intra- arterially and are thus formulated in a form suitable for intra-arterial administration. In some embodiments, the pharmaceutical compositions are administered intra-muscularly and are thus formulated in a form suitable for intra-muscular administration.

Proper formulation is dependent upon the route of administration selected. For injection, the agents of the compounds may be formulated into aqueous solutions, preferably in physiologically compatible buffers such as Hanks solution, Ringer's solution, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable carriers known in the art. Such carriers enable the compounds of the disclosure to be formulated as tablets, pills, dragees, capsules, liquids, gels, syrups, slurries, suspensions and the like, for oral ingestion by a patient to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, comprising lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration intranasally or by inhalation, the compounds for use according to the present disclosure may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

In addition to the formulations described above, the compounds may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt. A pharmaceutical carrier for hydrophobic compounds is a co-solvent system comprising benzyl alcohol, a non-polar surfactant, a water-miscible organic polymer, and an aqueous phase. The co-solvent system may be a VPD co-solvent system. VPD is a solution of 3% w/v benzyl alcohol, 8% w/v of the non-polar surfactant polysorbate 80, and 65% w/v polyethylene glycol 300, made up to volume in absolute ethanol. The VPD co-solvent system (VPD: 5 W) contains VPD diluted 1:1 with a 5% dextrose in water solution. This co-solvent system dissolves hydrophobic compounds well, and itself produces low toxicity upon systemic administration. The proportions of a co-solvent system may be suitably varied without destroying its solubility and toxicity characteristics. Furthermore, the identity of the co-solvent components may be varied: for example, other low-toxicity non-polar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity due to the toxic nature of DMSO. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutically acceptable formulations can contain a compound, or a salt or solvate thereof, in an amount of about 50 mg, about 100 mg, about 150 mg, about 200 mg, about 250 mg, about 300 mg, about 350 mg, about 400 mg, about 450 mg, or about 500 mg. Additionally, the pharmaceutically acceptable formulations may contain a compound, or a salt or solvate thereof, in an amount from about 0.5 w/w % to about 95 w/w %, or from about 1 w/w % to about 95 w/w %, or from about 1 w/w % to about 75 w/w %, or from about 5 w/w % to about 75 w/w %, or from about 10 w/w % to about 75 w/w %, or from about 10 w/w % to about 50 w/w %.

There are provided, in some embodiments, kits for preventing relapse to AML in a patient comprising a therapeutic amount of IL-2 and an agent disclosed herein, a means for identifying the presence of mutant nucleophosmin 1 (NPM1) as described herein, and instructions for the use of said kit. There are provided, in some embodiments, kits for prolonging remission from AML in a patient comprising a therapeutic amount of IL-2 and an agent disclosed herein, a means for identifying the presence of mutant nucleophosmin 1 (NPM1) as described herein, and instructions for the use of said kit.

### EXAMPLES

Some aspects of the embodiments discussed above are disclosed in further detail in the following examples, which are not in any way intended to limit the scope of the present disclosure.

### Example 1

### Phase IV Re:MISSION Clinical Trial

A phase IV clinical trial using HDC/IL-2 in treating AML patients recruited 84 patients in CR, after the completion of consolidation chemotherapy. The trial was conducted in 30 centers in Europe (Re:MISSION Trial, nr EPC 2008-02). Patients received HDC/IL-2 for relapse prevention in ten 3-week cycles as described in detail in Martner, Anna, et al. “Role of natural killer cell subsets and natural cytotoxicity receptors for the outcome of immunotherapy in acute myeloid leukemia.” Oncoimmunology 5.1 (2016): e1041701, the entirety of which is hereby incorporated by reference.

A single-armed multicenter phase IV study (Re:Mission, NCT01347996, registered at www.clinicaltrials.gov) enrolled 84 patients (age 18-79) with AML in first CR. The patients received ten consecutive 21-day cycles of histamine dihydrochloride (HDC; Ceplene) in combination with low-dose IL-2 during 18 months or until relapse or death. The treatment continued for a total of 18 months or until the patients relapsed, died, discontinued therapy because of adverse events, withdrew consent, or became lost to follow-up. Cycles 1 to 3 comprised 3 weeks of treatment and 3 weeks off treatment, and in cycles 4 to 10 the off-treatment periods were extended to 6 weeks. In each cycle, patients in the treatment arm received HDC (Meda Pharma, Frankfurt, Germany) at 0.5 mg subcutaneous twice a day and human recombinant IL-2 (aldesleukin; 16 400 U/kg subcutaneous twice a day; Chiron Corporation, Emeryville, CA). After 18 months of treatment (HDC/IL-2 arm), all patients were followed for at least six additional months after the end of immunotherapy. The dosage, route of administration, exclusion criteria etc. were identical to those described for a previous phase III trial [Brune et al., Blood. 2006; 108(1):88-96, incorporated herein by reference]. All data collected in support of these objectives were analyzed for the populations as a whole and by subgroups according to patient age at enrolment (<60 and >60 years).

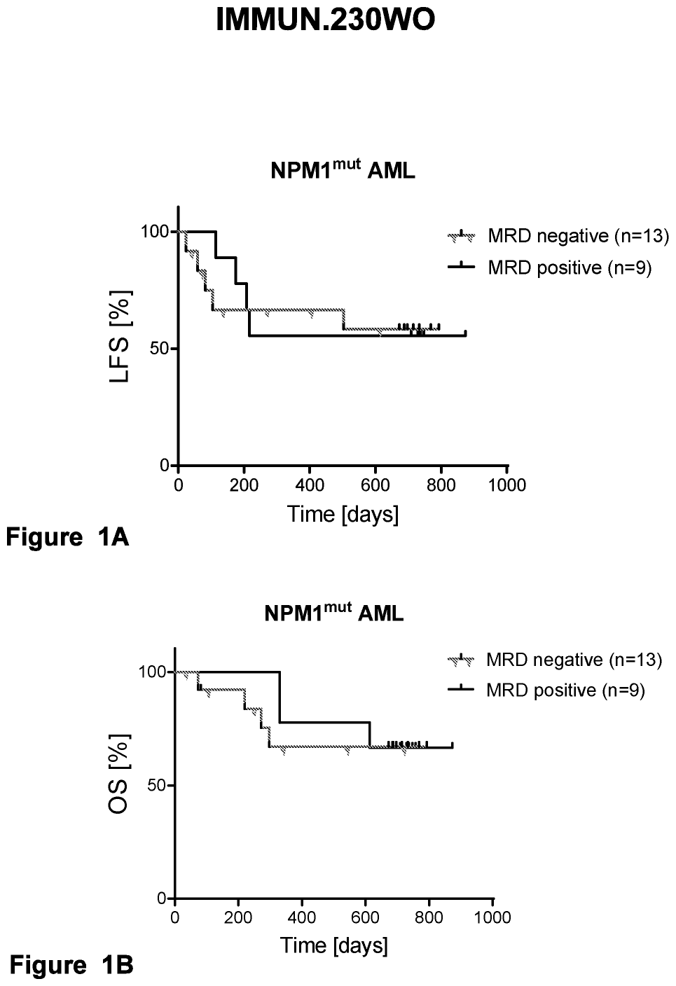

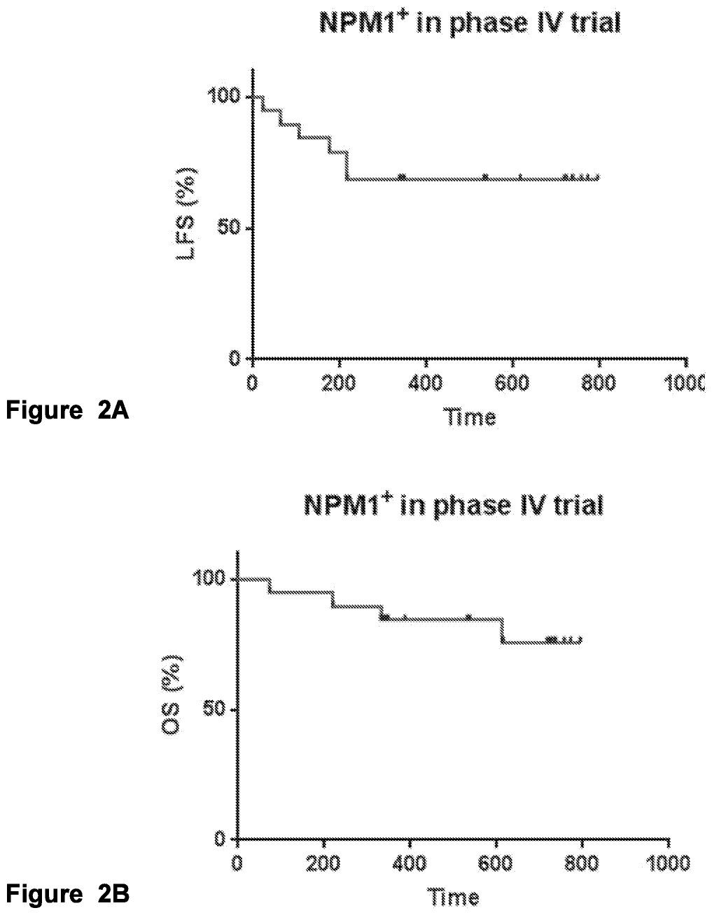

At diagnosis, bone marrow samples from these patients were independently analyzed according to routines at each participating center for presence of NPM1 mutations by the polymerase chain reaction assay, and 25 patients were classified as having NPM1c⁺ AML. Samples from 22 of these patients were additionally analyzed for MRD by RQPCR after the completion of consolidation chemotherapy, e.g. shortly before the onset of treatment with HDC/IL-2. The detailed characteristics of these patients are shown in Table 1. In 13/22 of these patients, transcripts of mutated NPM1 were undetectable at trial enrollment whereas such transcripts were detected in 9/22 patients (see column MRD+in Table 1). FIGS. 1A-1B show the outcome of these 22 patients in terms of leukemia-free survival (LFS, defined as the time from trial enrollment to relapse or death from any cause) and overall survival (OS, defined as time from enrollment to death), respectively. In 5/9 patients (56%) with presence of mutated NPM1 after the completion of treatment with HDC/IL-2 remained in CR, with 7/9 patients (78%) alive at >2 years. These results compare favorably with those presented by Ivey et al. (N Engl J Med. 2016 Feb. 4;374(5):422-33) where the rate of LFS at 2 years was 14% among NPM1MRD+patients at a corresponding stage of disease (i.e. after chemotherapy). FIGS. 2A-2B show the outcome of all patients with NPM1-mutated AML, with no other genetic aberrations, in terms of leukemia-free survival (LFS) and overall survival (OS), respectively. Collectively, these data compare favorably with historical controls, and demonstrate the efficacy of HDC/IL-2 administration in preventing relapse in NPM1-mutated patients, thereby increasing leukemia-free survival and overall survival.

TABLE 1
DETAILED CHARACTERISTICS OF 22 PHASE
IV CLINICAL TRIAL
PATIENTS WITH NPM1-MUTATION

| | OS in | Event | LFS in | Event | | | |
|---|---|---|---|---|---|---|---|
| MRD+ | days | Death | days | Relapse | FLT3 | Age | sex |
| yes | 874 | 0 | 874 | 0 | neg | 64.2 | M |
| yes | 748 | 0 | 748 | 0 | neg | 53.9 | M |
| yes | 737 | 0 | 737 | 0 | neg | 61.7 | F |
| yes | 729 | 0 | 729 | 0 | neg | 51.5 | F |
| yes | 709 | 0 | 709 | 0 | neg | 73.9 | F |
| yes | 330 | 1 | 216 | 1 | neg | 75.8 | F |
| yes | 758 | 0 | 208 | 1 | neg | 52.7 | F |
| yes | 613 | 1 | 175 | 1 | neg | 59.4 | F |
| yes | 330 | 1 | 115 | 1 | pos | 76.3 | M |
| no | 793 | 0 | 793 | 0 | neg | 66 | M |
| no | 769 | 0 | 769 | 0 | neg | 51.5 | M |
| no | 733 | 0 | 733 | 0 | neg | 70.4 | F |
| no | 715 | 0 | 715 | 0 | neg | 57.9 | M |
| no | 697 | 0 | 697 | 0 | neg | 65.3 | F |
| no | 687 | 0 | 687 | 0 | neg | 54.4 | M |
| no | 673 | 0 | 673 | 0 | neg | 27.6 | M |
| no | 685 | 0 | 503 | 1 | neg | 52.5 | F |
| no | 220 | 1 | 105 | 1 | neg | 66.3 | M |
| no | 272 | 1 | 91 | 1 | neg | 60.5 | M |
| no | 82 | 0 | 82 | 1 | pos | 63.5 | M |
| no | 297 | 1 | 58 | 1 | pos | 53.1 | F |
| no | 73 | 1 | 23 | 1 | neg | 61.4 | M |

### Example 2

### Second Phase IV Trial—MRD Trial

A second phase IV trial (“the MRD trial”) was performed in centers in Germany and Austria. Forty patients with confirmed AML in first CR received HDC/IL-2 using the regimen of a previous phase III trial (Brune et al., Blood 2006) and the above-referenced phase IV Re:Mission trial described in Example 1. An aim of this Phase IV trial was to define the potential efficacy of treatment with HDC/IL-2 on preventing the de novo occurrence of leukemic cells, and a primary endpoint was the re-appearance of leukemic cells in blood or bone marrow in patients who were MRD negative when they entered the trial.

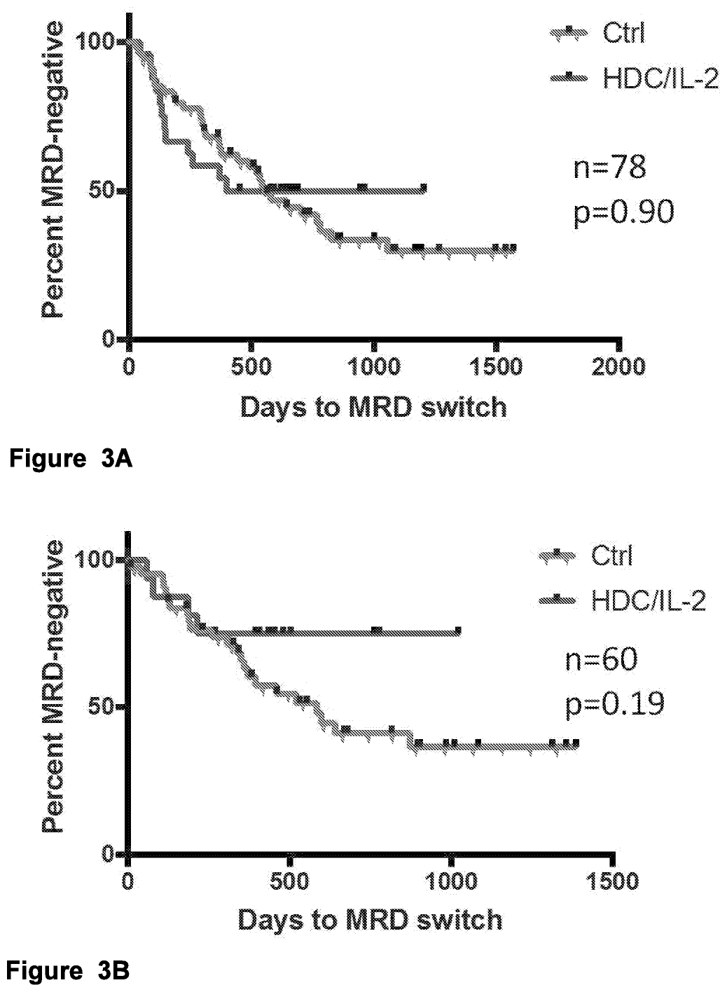

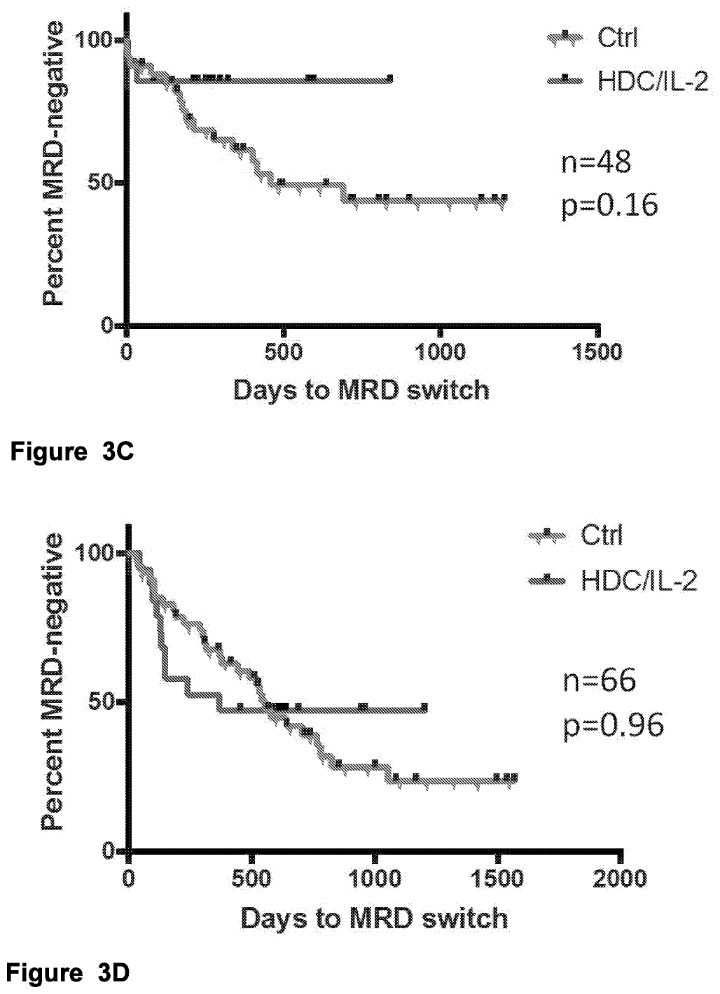

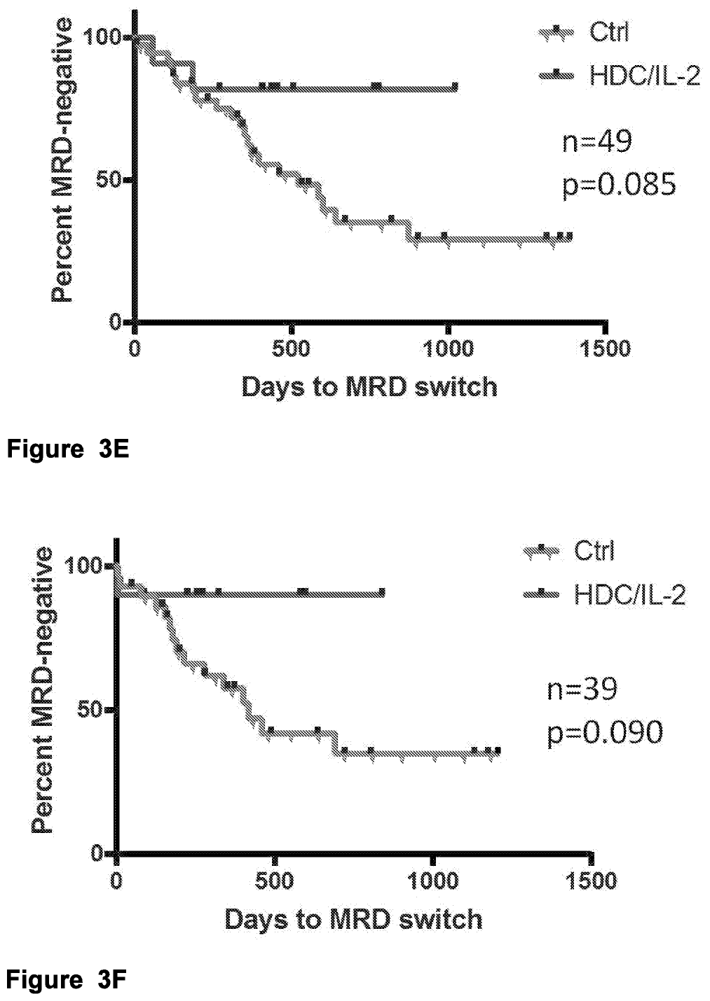

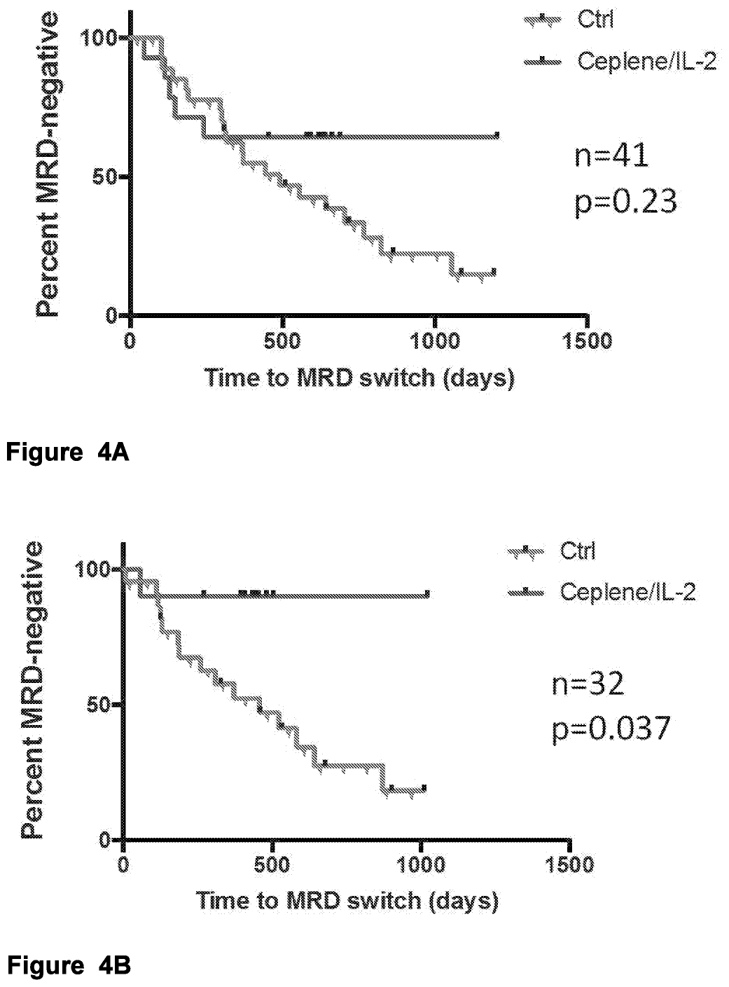

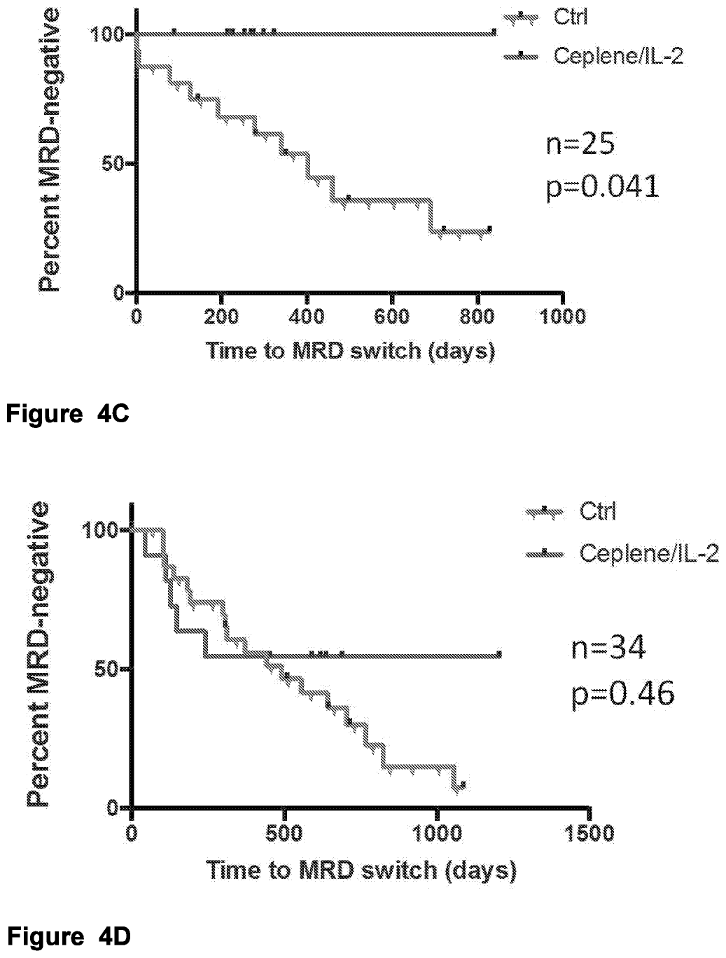

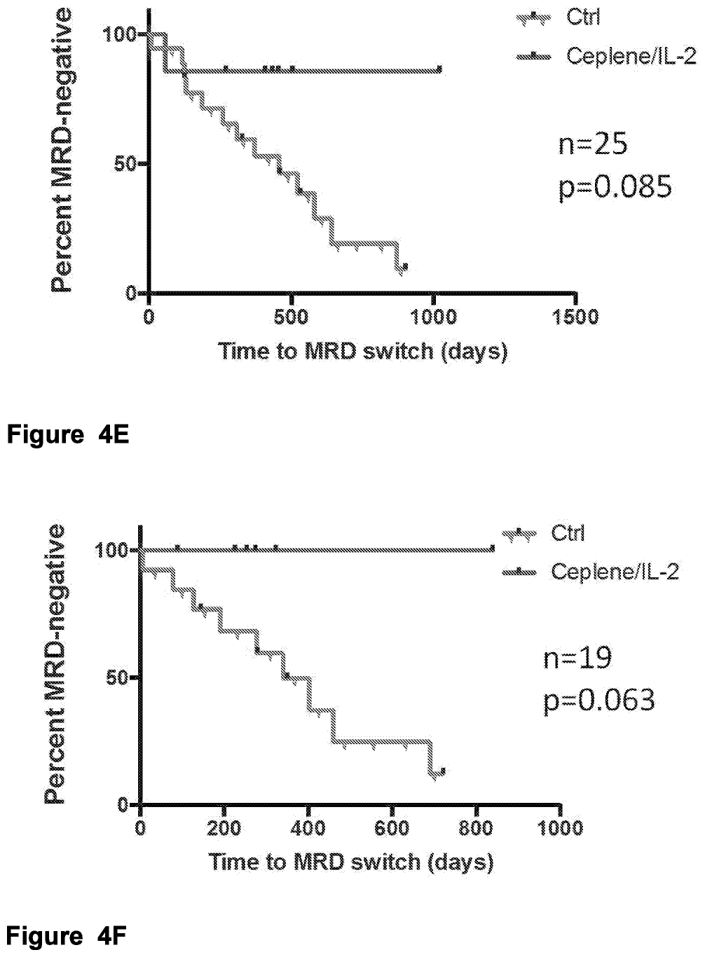

The results presented in FIGS. 3-4 demonstrate that HDC/IL-2 administration exerts measurable anti-leukemic efficacy, in terms of preventing the re-appearance of leukemia, in NPM1 mutant patients. The results achieved in patients treated with HDC/IL-2 were compared with those observed in age- and risk-matched contemporary historical control patients within the participating institutions. The interpretation is complicated by the fact that the control patients had received 3-4 times more anti-leukemic chemotherapy prior to inclusion in this trial than those who were treated with HDC/IL-2 (which may influence the risk of early molecular relapse and may skew the results in favor of the control arm, in particular with regards to early events). Tables 2 and 3 depict the demographics of the MRD Trial, where the groups compared in the Figures herein are indicated in italics and the differences in previous anti-leukemic chemotherapy are indicated in bold text. To control for these differences, landmark analyses were performed within the approved indication (in Europe, i.e. patients in first complete remission below the age of 60) and in all patients. The results (illustrating the time from inclusion to the first appearance of leukemic cells in blood) were compared with those obtained in matched historical controls from the participating centers. A prolongation of the appearance of leukemia is thus indicative of anti-leukemic activity of HDC/IL-2 vs. control in these patients. FIGS. 3 and 4 depict Kaplan-Meier curves showing days to MRD switch from negative to positive with and without landmark analysis for patients with NPM1-mutation. FIG. 3A shows results in all patients and their matched controls. FIG. 3B shows corresponding results with landmark analysis at 6 months, and FIG. 3C shows corresponding results with landmark analysis at 12 months. FIGS. 3D-F show corresponding results (i.e. no landmark (D), landmark at 6 months (E) and landmark at 12 months (F)) in the subgroup of patients with NPM1-mutation that did not receive low dose chemotherapy as maintenance (which is typically not practiced in most countries). FIG. 4 A-F show the results of FIG. 3 A-F for patients below 60 years of age with NPM1-mutation. Collectively, these results show that HDC/IL-2 prevented late (i.e., after 6 months or more) re-appearance of leukemic cells, thus demonstrating that the treatment exerts anti-leukemic activity against NPM1-positive AML cells in vivo.

TABLE 2
PATIENT DEMOGRAPHICS, ALL PATIENTS IN MRD TRIAL

| | CEP | | CEPmatch | | CON | | CEPnonmatch | |
|---|---|---|---|---|---|---|---|---|
| | (N = 75) | | (N = 40) | | (N = 74) | | (N = 35) | |
| Age (years) | | | | | | | | |
| n/nmiss | 75/0 | | 40/0 | | 74/0 | | 35/0 | |
| Mean (SD) | 54.92 | (15.22) | 53.83 | (14.73) | 56.45 | (13.92) | 56.17 | (15.89) |
| Median | 58.00 | | 57.00 | | 58.00 | | 59.00 | |
| Q1, Q3 | 42.00, 68.00 | | 43.00, 65.50 | | 46.00, 70.00 | | 42.00, 69.00 | |
| Min, Max | 19.0, 79.0 | | 19.0, 78.0 | | 23.0, 77.0 | | 21.0, 79.0 | |
| Age class | | | | | | | | |
| ≤60 years | 41 | (54.7%) | 23 | (57.5%) | 40 | (54.1%) | 18 | (51.4%) |
| >60 years | 34 | (45.3%) | 17 | (42.5%) | 34 | (45.9%) | 17 | (48.6%) |
| Sex | | | | | | | | |
| Female | 33 | (44.0%) | 19 | (47.5%) | 36 | (51.3%) | 14 | (40.0%) |
| Male | 42 | (56.0%) | 21 | (52.5%) | 38 | (48.7%) | 21 | (60.0%) |

Abbreviations: n/nmiss, number of subjects with evaluable/missing data;
SD, standard deviation; Q1/Q3, quartiles.
Percentages are based on the number of subjects in the respective analysis set.
Age is the age at diagnosis.

TABLE 3
AML HISTORY AND PREVIOUS AML TREATMENT, ALL PATIENTS

| | | | |
|---|---|---|---|
| CEP | CEPmatch | CON | CEPnonmatch |
| (N = 75) | (N = 40) | (N = 74) | (N = 35) |

No. of cycles
induction therapy

| 1 | 65 | (86.7%) | 33 | (82.5%) | 15 | (20.3%) | 32 | (91.4%) |
|---|---|---|---|---|---|---|---|---|
| 2 | 10 | (13.3%) | 7 | (17.5%) | 59 | (79.7%) | 3 | (8.6%) |
| ≥3 | 0 | (0.0%) | 0 | (0.0%) | 0 | (0.0%) | 0 | (0.0%) |
| n/nmiss | 75/0 | | 40/0 | | 74/0 | | 35/0 | |
| Mean (SD) | 1.33 | (0.34) | 1.18 | (0.38) | 1.80 | (0.40) | 1.09 | (0.28) |
| Median | 1.00 | | 1.00 | | 2.00 | | 1.00 | |
| Q1, Q3 | 1.00, 1.00 | | 1.00, 1.00 | | 2.00, 2.00 | | 1.00, 1.00 | |
| Min, Max | 1.0, 2.0 | | 1.0, 2.0 | | 1.0, 2.0 | | 1.0, 2.0 | |
| No. of cycles | | | | | | | | |
| consolidation therapy | | | | | | | | |
| 0 | 4 | (5.3%) | 0 | (0.0%) | 1 | (1.3%) | 4 | (11.4%) |
| 1 | 43 | (57.3%) | 27 | (67.5%) | 7 | (9.5%) | 16 | (45.7%) |
| 2 | 1 | (1.3%) | 1 | (2.5%) | 1 | (1.3%) | 0 | (0.0%) |
| 3 | 9 | (12.0%) | 6 | (15.0%) | 54 | (73.0%) | 3 | (8.6%) |
| ≥4 | 18 | (24.0%) | 6 | (15.0%) | 11 | (14.9%) | 12 | (34.3%) |
| n/nmiss | 75/0 | | 40/0 | | 74/0 | | 35/0 | |
| Mean (SD) | 1.93 | (1.40) | 1.78 | (1.19) | 2.91 | (0.81) | 2.11 | (1.60) |
| Median | 1.00 | | 1.00 | | 3.00 | | 1.00 | |
| Q1, Q3 | 1.00, 3.00 | | 1.00, 3.00 | | 3.00, 3.00 | | 1.00, 4.00 | |
| Min, Max | 0.0, 5.0 | | 1.0, 4.0 | | 0.0, 4.0 | | 0.0, 5.0 | |
| No. of cycles | | | | | | | | |
| LDC therapy | | | | | | | | |
| 0 | 65 | (86.7%) | 32 | (80%) | 62 | (83.8%) | 33 | (94.3%) |
| 1-3 | 7 | (9.3%) | 5 | (12.5%) | 5 | (6.8%) | 2 | (5.7%) |
| 4-6 | 1 | (1.3%) | 1 | (2.5%) | 0 | (0.0%) | 0 | (0.0%) |
| 7-9 | 1 | (1.3%) | 1 | (2.5%) | 0 | (0.0%) | 0 | (0.0%) |
| 10-12 | 0 | (0.0%) | 0 | (0.0%) | 4 | (5.4%) | 0 | (0.0%) |
| ≥13 | 1 | (1.3%) | 1 | (2.5%) | 3 | (4.0%) | 0 | (0.0%) |
| n/nmiss | 75/0 | | 40/0 | | 74/0 | | 35/0 | |
| Mean (SD) | 0.55 | (2.04) | 0.93 | (2.71) | 1.66 | (5.54) | 0.11 | (0.53) |

| | | | | |
|---|---|---|---|---|
| Median | 0.00 | 0.00 | 0.00 | 0.00 |
| Q1, Q3 | 0.00, 0.00 | 0.00, 0.00 | 0.00, 0.00 | 0.00, 0.00 |
| Min, Max | 0.0, 14.0 | 0.0, 14.0 | 0.0, 36.0 | 0.0, 3.0 |

Percentages are based on the number of subjects in the respective analysis set.

In at least some of the previously described embodiments, one or more elements used in some embodiments can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including patents, patent applications, papers, text books, and the like, and the references cited herein, to the extent that they are not already, are hereby incorporated by reference in their entirety. In the event that one or more of the incorporated literature and similar materials differ from or contradict this application, including but not limited to defined terms, term usage, described techniques, or the like, this application controls.

What is claimed is:

1. A method for improving a survival rate of patients having acute myeloid leukemia (AML), comprising the steps of:
(a) identifying the presence of mutant nucleophosmin 1 (NPM1) in a patient having AML; and
(b) administering to a patient identified as having a mutant NPM1 in step (a) a therapeutically effective amount of IL2 and a therapeutically effective amount of an agent selected from the group consisting of histamine, a histamine structural analog having H₂-receptor activities, an endogenous histamine releasing preparation, a non-histamine derivative H₂-receptor agonist, and a combination thereof,
wherein the administration of said IL-2 and said agent results in an increase in said survival rate of said patients compared to the untreated patients.

2. A method of preventing and/or delaying the onset of relapse to acute myeloid leukemia (AML) in a patient in complete remission (CR) from AML, comprising the steps of:
(a) identifying the presence of mutant nucleophosmin 1 (NPM1) in a patient in complete remission (CR) from AML; and
(b) administering to a patient identified as having a mutant NPM1 in step (a) a therapeutically effective amount of IL2 and a therapeutically effective amount of an agent selected from the group consisting of histamine, a histamine structural analog having H₂-receptor activities, an endogenous histamine releasing preparation, a non-histamine derivative H₂-receptor agonist, and a combination thereof,
thereby preventing and/or delaying the onset of relapse to AML in said patient.

3. A method of prolonging remission from acute myeloid leukemia (AML), comprising the steps of:
(a) identifying the presence of mutant nucleophosmin 1 (NPM1) in a patient in remission from AML; and
(b) administering to a patient identified as having a mutant NPM1 in step (a) a therapeutically effective amount of IL2 and a therapeutically effective amount of an agent selected from the group consisting of histamine, a histamine structural analog having H₂-receptor activities, an endogenous histamine releasing preparation, a non-histamine derivative H₂-receptor agonist, and a combination thereof,
thereby prolonging remission from AML in said patient.

4. A method for improving a survival rate of patients having acute myeloid leukemia (AML), comprising the steps of:
(a) acquiring knowledge of the presence of one or more molecular alterations in a biological sample from an AML patient, wherein said one or more molecular alterations comprises the presence of mutant nucleophosmin 1 (NPM1); and
(b) for a patient known to have a mutant NPM1 in step (a), administering to the patient a therapeutically effective amount of IL2 and a therapeutically effective amount of an agent selected from the group consisting of histamine, a histamine structural analog having H₂-receptor activities, an endogenous histamine releasing preparation, a non-histamine derivative H₂-receptor agonist, and a combination thereof,
wherein the administration of said IL-2 and said agent results in an increase in said survival rate of said patients compared to the untreated patients.

5. A method of preventing and/or delaying the onset of relapse to acute myeloid leukemia (AML) in a patient in complete remission (CR) from AML, comprising the steps of:
(a) acquiring knowledge of the presence of one or more molecular alterations in a biological sample from an AML patient, wherein said one or more molecular alterations comprises the presence of mutant nucleophosmin 1 (NPM1); and
(b) for a patient known to have a mutant NPM1 in step (a), administering to the patient a therapeutically effective amount of IL2 and a therapeutically effective amount of an agent selected from the group consisting of histamine, a histamine structural analog having H₂-receptor activities, an endogenous histamine releasing preparation, a non-histamine derivative H₂-receptor agonist, and a combination thereof,
thereby preventing and/or delaying the onset of relapse to AML in said patient known to have a mutant NPM1.

6. A method of prolonging the remission from acute myeloid leukemia (AML), comprising the steps of:
(a) acquiring knowledge of the presence of one or more molecular alterations in a biological sample from an AML patient, wherein said one or more molecular alterations comprises the presence of mutant nucleophosmin 1 (NPM1); and
(b) for a patient known to have a mutant NPM1 in step (a), administering to the patient a therapeutically effective amount of IL2 and a therapeutically effective amount of an agent selected from the group consisting of histamine, a histamine structural analog having H₂-receptor activities, an endogenous histamine releasing preparation, a non-histamine derivative H₂-receptor agonist, and a combination thereof,
thereby prolonging remission from AML in said patient known to have a mutant NPM1.

7. The method of any one of claims 1-6, wherein the patient is in complete remission (CR) from AML, wherein the CR comprises less than 5% blast cells in normocellular bone marrow and an absence of extramedullary leukemia.

8. The method of any one of claims 1-7, wherein said agent is administered twice a day.

9. The method of any one of claims 1-8, wherein said agent is administered in an amount of about 1 mg/day to about 10 mg/day.

10. The method of any one of claims 1-9, wherein said agent is histamine.

11. The method of claim 10, wherein said histamine is histamine dihydrochloride.

12. The method of claim 10, wherein said histamine is histamine diphosphate.

13. The method of any one of claims 10-12, wherein the histamine is administered at 0.5 mg twice a day.

14. The method of any one of claims 1-13, wherein said IL-2 is administered in an amount of about 5,000 U/kg/day to about 300,000 U/kg/day.

15. The method of any one of claims 1-14, wherein said IL-2 is administered at a dosage of 16,400 U/kg twice a day.

16. The method of any one of claims 1-15, wherein said agent and IL-2 are administered on the same days.

17. The method of any one of claims 1-16, wherein said agent and IL-2 are administered together.

18. The method of any one of claims 1-17, wherein the administration of said agent and said IL-2 is performed simultaneously.

19. The method of any one of claims 1-17, wherein said agent and IL-2 are administered separately.

20. The method of any one of claims 1-19, wherein the administration of said agent and the administration of said IL-2 are performed within 24 hours.

21. The method of any one of claims 1-20, wherein the administration of said agent and said IL-2 is accomplished by one or more of intramuscular injection, subcutaneous injection, intradermal injection, intravenous injection, implantation infusion device, inhalation, and transdermal diffusion.

22. The method of any one of claims 1-21, wherein the administration of said agent and said IL-2 is accomplished by subcutaneous injection.

23. The method of any one of claims 1-22, wherein said agent and said IL-2 is administered once per day.

24. The method of any one of claims 1-23, wherein said agent and said IL-2 is administered for at least one cycle.

25. The method of any one of claims 1-24, wherein said agent and said IL-2 is administered for at least two cycles.

26. The method of any one of claims 1-25, wherein said agent and said IL-2 is administered for six cycles.

27. The method of any one of claims 24-26, wherein the at least one cycle comprises 21 consecutive days of treatment.

28. The method of any one of claims 24-27, wherein an interval between two treatment cycles is at least two weeks.

29. The method of claim 28, wherein the interval is at least three weeks.

30. The method of claim 28, wherein the interval is at least six weeks.

31. The method of any one of claims 1-30, wherein the patient has a de novo AML.

32. The method of any one of claims 1-30, wherein the patient has a secondary AML.

33. The method of any one of claims 1-32, wherein the patient has recurrent, relapsing or refractory AML.

34. The method of claim 33, wherein the recurrent or relapsing AML is caused by minimal residual disease (MRD) or leukemic stem cells.

35. The method of any one of claims 1-34, wherein the patient has already undergone 2 or more rounds of chemotherapy.

36. The method of any one of claims 1-35, wherein the patient has already undergone 4 or more rounds of chemotherapy.

37. The method of any one of claims 1-36, wherein said patient is undergoing immunotherapy for relapse prevention.

38. The method of any one of claims 1-37, wherein the patient has experienced a partial response or complete response, is in remission, is asymptomatic, has a low number of abnormal cells and/or has a non-detectable disease based on one or more of the following: (i) a total body leukemia burden below approximately 109 cells and/or less than 5% blasts in the marrow and/or no signs or symptoms of leukemia; (ii) a greater than 25% reduction in the serum protein M level; (iii) a greater than 50% reduction in the serum protein M level; (iv) 10% or more plasma cells in the bone marrow, but does not meet the criteria for multiple myeloma (MM); (v) serum M proteins levels greater than or equal to 3 g/dL; (vi) 10% or more plasma cells in the bone marrow with no evidence of end-organ damage; (vii) serum M protein levels greater than or equal to 3 g/dL and has 10% or more plasma cells in the bone marrow; (viii) serum M protein levels greater than or equal to 3 g/dL and has 10% or more plasma cells in the bone marrow and no evidence of end-organ damage; and (ix) less than 10% plasma cells in the bone marrow.

39. The method of any one of claims 1-38, wherein the patient has completed induction chemotherapy.

40. The method of any one of claims 1-39, wherein the patient is a patient who relapses from complete remission of AML after induction chemotherapy.

41. The method of any one of claims 1-40, wherein the patient has completed induction and consolidation chemotherapy.

42. The method of any one of claims 1-41, wherein said administration of said IL-2 and said agent begins the same day after consolidation chemotherapy is completed.

43. The method of any one of claims 1-41, wherein said administration of said IL-2 and said agent begins between about 1 day and about 300 days after consolidation chemotherapy is completed.

44. The method of any one of claims 1-41, wherein said administration of said IL-2 and said agent begins about 200 days after consolidation chemotherapy is completed.

45. The method of any one of claims 1-41, wherein said administration of said IL-2 and said agent begins about 100 days after consolidation chemotherapy is completed.

46. The method of any one of claims 1-41, wherein said administration of said IL-2 and said agent begins about 50 days after consolidation chemotherapy is completed.

47. The method of any one of claims 1-46, wherein said administration of said IL-2 and said agent results in an increase of at least 30% in a survival rate of said patients compared to the untreated patients.

48. The method of any one of claims 1-47, wherein said administration of said IL-2 and said agent results in an increase of at least 30% in a survival rate of said patients compared to the untreated patients.

49. The method of any one of claims 1-48, wherein said administration of said IL-2 and said agent results in an increase of at least 50% in a survival rate of said patients compared to the untreated patients.

50. The method of any one of claims 1, 4, and 7-49, wherein said survival rate is leukemia-free survival rate.

51. The method of any one of claims 1, 4, and 7-49, wherein said survival rate is overall survival rate.

52. The method of any one of claims 1-51, wherein said administration of said IL-2 and said agent delays relapse of AML of said patients by at least 3 months compared to the untreated patients.

53. The method of any one of claims 1-52, wherein said administration of said IL-2 and said agent delays relapse of AML of said patients by at least 6 months compared to the untreated patients.

54. The method of any one of claims 1-53, wherein said administration of said IL-2 and said agent delays relapse of AML of said patients by at least 12 months compared to the untreated patients.

55. The method of any one of claims 2, 5, and 7-54, wherein relapse comprises at least 5% blast cells in the bone marrow.

56. The method of any one of claims 2, 5, and 7-54, wherein relapse comprises extramedullary leukemia.

57. The method of any one of claims 1-56, wherein said administration of said IL-2 and said agent prolongs remission from AML of said patients by at least 3 months compared to the untreated patients.

58. The method of any one of claims 1-57, wherein said administration of said IL-2 and said agent prolongs remission from AML of said patients by at least 6 months compared to the untreated patients.

59. The method of any one of claims 1-58, wherein said administration of said IL-2 and said agent prolongs remission from AML of said patients by at least 12 months compared to the untreated patients.

60. The method of any one of claims 4-59, wherein said knowledge is acquired from an analytical assay selected from the group consisting of nucleic acid sequencing, polypeptide sequencing, restriction digestion, capillary electrophoresis, nucleic acid amplification-based assays, nucleic acid hybridization assay, comparative genomic hybridization, real-time PCR, quantitative reverse transcription PCR (qRT-PCR), PCR-RFLP assay, HPLC, mass-spectrometric genotyping, fluorescent in-situ hybridization (FISH), next generation sequencing (NGS), a kinase activity assay, and any combination thereof.

61. The method of any one of claims 4-59, wherein said knowledge is acquired from an antibody-based assay selected from ELISA, immunohistochemistry, western blotting, mass spectrometry, flow cytometry, protein-microarray, immunofluorescence, a multiplex detection assay, or any combination thereof.

62. The method of any one of claims 4-59, wherein said knowledge is acquired from immunohistochemistry.

63. The method of any one of claims 1-62, wherein the patient's leukemic cells have a normal karyotype.

64. The method of any one of claims 1-63, wherein the presence of the mutant NPM1 is determined by identifying a patient nucleic acid encoding the mutant NPM1.

65. The method of claim 64, wherein identifying a patient nucleic acid encoding the mutant NPM1 comprises amplification of at least a portion of exon 12 of NPM1.

66. The method of claim 65, wherein said amplification comprises the polymerase chain reaction (PCR).

67. The method of claim 66, wherein said PCR is real-time PCR (RT-PCR).

68. The method of any one of claims 64-67, wherein said patient nucleic acid is obtained from an acellular body fluid of said patient.

69. The method of claim 68, wherein said acellular body fluid is serum or plasma.

70. The method of any one of claims 64-69, wherein said patient nucleic acid is genomic DNA.

71. The method of any one of claims 64-69, wherein said patient nucleic acid is mRNA.

72. The method of any one of claims 1-63, wherein identifying a patient nucleic acid encoding the mutant NPM1 comprises using an oligonucleotide probe complimentary to a portion of exon 12 of NPM1.

73. The method of claim 72, wherein the oligonucleotide probe comprises a label.

74. The method of claim 73, wherein the label is fluorescent.

75. The method of any one of claims 1-74, wherein the mutant NPM1 comprises one or more mutations in exon 12 NPM1 that cause cytoplasmic location of NPM1 protein

76. The method of any one of claims 1-75, wherein the mutant NPM1 comprises one or more of the following NPM1 mutations: Mutation A, Mutation B, Mutation C, Mutation D, Mutation E or Mutation F.

77. The method of any one of claims 1-75, wherein the mutant NPM1 comprises one or more of the NPM1 mutations selected from the group consisting of: Mutation A, Mutation B, Mutation C, Mutation D, Mutation E, Mutation F, Mutation E*, Mutation G*, Mutation H*, Mutation J, Mutation L, Mutation K, Mutation M, Mutation N, Mutation O, Mutation P, Mutation Q, Mutation Gm, Mutation Km, Mutation Lm, Mutation Nm, Mutation Om, Mutation Qm, Mutation 1, Mutation 3, Mutation 4, Mutation 6, Mutation 7, Mutation 12, Mutation 13, Mutation 10, Mutation 14, Mutation G+, Mutation H+, Mutation I+, Mutation J+, Mutation I, and a combination thereof.

78. The method of any one of claims 1-75, wherein the mutant NPM1 comprises a signal motif of nuclear export (NES) in exon 12 of NPM1, wherein the NES comprises the amino acid sequence YxxxYxxYxY, wherein Y is a hydrophobic amino acid selected from the group consisting of leucine, isoleucine, methionine, valine, phenylalanine, and wherein x can be any amino acid.

79. The method of any one of claims 1-63, wherein the presence of the mutant NPM1 is determined by identifying mutant NPM1 protein in patient cells.

80. The method of claim 79, wherein the mutant NPM1 protein is identified in the cells by identifying NPM1 protein in cytoplasm of the cells.

81. The method of claim 80, wherein the mutant NPM1 protein is identified in cytoplasm of the cells immunohistochemically.

82. The method of claim 79, wherein the mutant NPM1 protein is identified in the cells with an antibody that selectively binds to the mutant NPM1 protein but not a wildtype NPM1 protein.
